(19) United States
(12) Patent Application Publication
(10) Pub. No.: US 2013/0078142 A1
(43) Pub. Date: Mar. 28, 2013

(54) Method and Apparatus for Sterilization of Medical Instruments and Devices by Ultraviolet Sterilization

(71) Applicant: Eugene I. Gordon, Mountainside, NJ (US)

(72) Inventor: Eugene I. Gordon, Mountainside, NJ (US)

(21) Appl. No.: 13/622,726

(22) Filed: Sep. 19, 2012

Related U.S. Application Data
(60) Provisional application No. 61/537,731, filed on Sep. 22, 2011.

(51) Int. Cl. A61L 2/10; B65D 25/00; B65D 33/00

(52) U.S. Cl. 422/24; 383/105; 220/660

(57) ABSTRACT

An object is sterilized with ultraviolet-C (UV-C) radiation having a wavelength from about 235 nm to about 295 nm. An advantageous wavelength for the UV-C radiation is about 253.7 nm. The object is inserted into a container, which is then sealed. At least a portion of the container is substantially transparent to UV-C radiation over the wavelengths of interest. The container and object are placed into a UV-C irradiation device and are irradiated for an exposure time with UV-C radiation having a predetermined intensity. The exposure time is determined such that a predetermined portion of user-specified pathogens disposed on the object is inactivated. Technical sterilization (99.9999% inactivation) can be attained with relatively short exposure times. The container can be a flexible pouch or a rigid kit. Suitable materials that are substantially transparent to UV-C radiation over the wavelengths of interest include quartz, borosilicate glass, cyclic olefin copolymer, and fluoropolymer.

This application claims the benefit of U.S. Provisional Application No. 61/537,731 filed Sep. 22, 2011, which is incorporated herein by reference.

### BACKGROUND OF THE INVENTION

The present invention relates generally to sterilization, and more particularly to method and apparatus for sterilization of medical instruments and devices by ultraviolet radiation.

Sterilization of medical instruments and devices (in particular, surgical instruments) is a critical process for medical procedures; for example, newly-manufactured surgical instruments need to be sterilized prior to their first use, and previously-used surgical instruments need to be sterilized prior to their next use. Surgical instrument sterilizers based on various operating principles are currently available; examples include gamma radiation sterilizers, gas-based sterilizers, and steam-based autoclave sterilizers. Each of these has specific advantages and disadvantages.

Gamma radiation sterilizers, for example, can be effective; however, they are expensive and require long process times. Other surgical instrument sterilizers operate at room temperature and use various gases for sterilization. They have the disadvantages of long process times (typically at least 30 to 60 minutes), limited effectiveness for instruments with internal volumes (lumens), and degradation of some materials; furthermore, in addition to electric power, these systems require vacuum pumps. The ethylene oxide (ETO) sterilizer has a cycle time of 15 hours, is explosive and poisonous, and is outlawed in some states. Most of the currently deployed central processing systems use vaporized hydrogen peroxide (VH₂O₂); some use a combination of ozone (O₃) and water vapor (H₂O) or a combination of O₃ and VH₂O₂. A combination of O₃ and a hydrocarbon derivative such as isopropyl alcohol can shorten the cycle time to approximately two minutes; at least two cycles are needed to meet United States Food and Drug Administration (FDA) requirements.

In steam autoclaves, surgical instruments are sterilized with pressurized steam, typically at a temperature of 121° C. Steam autoclaves can sterilize instruments with external surfaces only (“coupon devices”) as well as those with internal volumes that have openings to the outside (“lumen devices”). The sterilization time itself can be as short as six minutes, but the complete process (including double wrapping the instruments with linen, warm-up, steam sterilization, cool-down, and unwrapping) takes about an hour. The steam process can also produce damage or compromise the sharpness of some instruments and thereby requires periodic steps to restore them. Furthermore, in addition to electric power, steam autoclaves require a water supply and plumbing for the water disposal. Nevertheless, despite these drawbacks, steam autoclaves are the most common sterilizer in use.

Fast effective sterilization would be advantageous to improve productivity and to provide low sterilization cost per instrument cycle. In many circumstances in a hospital, it would also be highly desirable to fully sterilize in minutes a compromised surgical instrument for emergency use during surgery. A similar procedure is also advantageous for sterilizing laboratory instruments. Such expedited capability, known as “flash sterilization”, is currently available only with steam autoclaves operated with unwrapped instruments at a temperature of 132° C., as illustrated in Table 1 below.

TABLE 1

| STEAM | | | |
|---|---|---|---|
| STERILIZER | CYCLE | STEAM | STERILIZATION |
| CYCLE | TEMPERATURE | PRESSURE | CYCLE TIME |
| Basic autoclave | 121° C. (250° F.) | 15 psi | 15 min |
| With heavily | 132° C. (270° F.) | 30 psi | 10 min |
| wrapped items | | | |
| With unwrapped | 132° C. (270° F.) | 30 psi | 3 min |
| items | | | |

Unwrapped instruments, therefore, can be sterilized at an elevated temperature of 132° C. with a 3-minute sterilization cycle; including time for warm-up and cool-down, flash sterilization with a steam autoclave has a full process cycle of typically 6-9 minutes. This process, however, has the potential serious deficiency that the sterilization of the instrument may be compromised by handling without wrapping and by exposure to room air, which is never sterile.

The instrument is certainly no longer totally free of contamination by the time it reaches the surgeon's hands. Flash sterilization is considered to be an emergency procedure for pathogen-compromised, surgical instruments, although often it is used in non-emergency situations as a money-saving shortcut. The high process temperature, furthermore, can shorten the lifetime of the instrument and increase the frequency for maintenance.

Even with wrapped instruments, there are still issues with maintaining with absolute certainty the sterility of the instrument in the pouch or kit until use. The packaging itself, for example, can become contaminated. In surgical practice, it is common for unused instruments to be returned from an operating room to a sterile storage room many times. The outside of the package can become contaminated by blood, for example, in the operating room. Contaminants on the outside of the package can then contaminate the instrument when the package is finally opened for use. Some packages, such as the common sterile wrap-and-peel pouch, can become frayed through repeated handling, and the integrity of the packaging can become compromised.

Most surgical instruments are handled before they get into the surgeon's hands. Instruments are routinely handled in the process of removal from the sterilization system, followed by placement in a sterile pouch that is then sealed or followed by placement in an instrument kit that is then closed. These procedures can compromise sterility. Instruments can become contaminated from pathogens in the air or on gloves. By the time the sterilized surgical instrument is placed in the surgeon's hands for use, its sterility can be assumed to be compromised. Maintaining sterility is critical for surgery: open surgical sites can become infected by as few as 10 pathogens.

### BRIEF SUMMARY OF THE INVENTION

In an embodiment of the invention, an object is sterilized with ultraviolet-C radiation having a wavelength from about 235 nm to about 295 nm. The object is inserted into a container, which is then sealed. At least a portion of the container is substantially transparent to ultraviolet-C radiation having a wavelength from about 235 nm to about 295 nm. The container and object are then irradiated for an exposure time with ultraviolet-C radiation having a wavelength from about 235 nm to about 295 nm and having a predetermined intensity. The exposure time is determined such that a predetermined portion of user-specified pathogens disposed on the object prior to irradiation is inactivated. In an embodiment, the wavelength of the UV-C radiation is about 253.7 nm.

In an embodiment of the invention, the container is a flexible pouch fabricated from a material substantially transparent to ultraviolet-C radiation having a wavelength from about 235 nm to about 295 nm. Suitable materials include cyclic olefin copolymer and fluoropolymer. The pouch can be sealed with an adhesive or by thermal fusion. The pouch can also be mechanically sealed.

In an embodiment of the invention, the container is a rigid kit including a receptacle and a cover. The receptacle includes a receptacle plate fabricated from a material substantially transparent to ultraviolet-C radiation having a wavelength from about 235 nm to about 295 nm, a side wall sealed to the receptacle plate, and a receptacle opening opposed to the receptacle plate. The cover includes a cover plate fabricated from a material substantially transparent to ultraviolet-C radiation having a wavelength from about 235 nm to about 295 nm. The cover is configured to mechanically seal the receptacle opening such that the cover plate is opposed to the receptacle plate. Suitable materials for the receptacle plate and the cover plate include quartz, borosilicate glass, and fluoropolymer.

In an embodiment of the invention, the container is a rigid kit including a receptacle and a cover. The receptacle includes a first receptacle plate, a second receptacle plate opposed to the first receptacle plate, a side wall sealed to the first receptacle plate and to the second receptacle plate, and a receptacle opening opposed to a portion of the side wall. The first receptacle plate is fabricated from a material substantially transparent to ultraviolet-C radiation having a wavelength from about 235 nm to about 295 nm. The second receptacle plate is fabricated from a material substantially transparent to ultraviolet-C radiation having a wavelength from about 235 nm to about 295 nm. The cover is configured to mechanically seal the receptacle opening. Suitable materials for the first receptacle plate and the second receptacle plate include quartz, borosilicate glass, and fluoropolymer.

These and other advantages of the invention will be apparent to those of ordinary skill in the art by reference to the following detailed description and the accompanying drawings.

### BRIEF DESCRIPTION OF THE DRAWINGS

FIG. 1A-FIG. 1F show schematic views of ultraviolet irradiation devices;

FIG. 2 shows plots of transmission as a function of wavelength for several materials;

FIG. 3A-FIG. 3L show schematic views of instrument pouches;

FIG. 4A-FIG. 4L show schematic views of instrument kits;

FIG. 5A-FIG. 5F illustrate a method for sterilizing an object sealed within a pouch;

FIG. 6A-FIG. 6F illustrate a method for sterilizing an object sealed within a kit;

FIG. 7A-FIG. 7D show schematic views of an object with a pivot joint;

FIG. 8A-FIG. 8C show schematic views of an object with a tubular structure; and

FIG. 9A-FIG. 9C show schematic views of an object with a cavity.

### DETAILED DESCRIPTION

Ultraviolet irradiation in a specific range of wavelengths (discussed below) can be used to inactivate all pathogen types including, for example, anthrax and C. difficile endospores, S. aureus (antibiotic forms are also known as MRSA), smallpox, viral hemorrhagic fevers, pneumonic plague, glanders, tularemia, and drug-resistant tuberculosis. Pathogens that have a relatively thick cell wall, such as endospores, are more resistant to ultraviolet irradiation because the thick cell wall transmits less ultraviolet radiation; consequently, the ultraviolet radiation intensity inside the cell wall is reduced. With higher intensities or longer exposure times (or a combination of both higher intensities and longer exposure times), however, even the most resistant endospores are readily inactivated by ultraviolet irradiation.

The effectiveness of ultraviolet irradiation derives primarily from a narrow band of ultraviolet-C (UV-C) radiation about 60 nm wide centered at a wavelength of about 260 nm; that is, wavelengths ranging from about 235 nm to about 295 nm. The UV-C radiation in that particular band acts by eliminating the ability of any given pathogen to reproduce through mitosis and potentially cause an infection. Eliminating the ability to undergo mitosis is called inactivation.

Radiation intensity is a measure of radiant power incident per unit area. If a pathogen is in the presence of UV-C radiation of a given wavelength for a given exposure time, the integral of the radiation intensity received by the pathogen over time determines the radiant energy exposure per unit area. The surface area of the pathogen defines the actual energy incident on and passing through the pathogen. Statistically the incident photons passing through the pathogen have a reasonable probability of being absorbed by a particular DNA molecule within the pathogen, breaking certain bonds within the DNA molecule. The DNA molecule loses its ability to trigger mitosis in the pathogen, and the pathogen loses its ability to multiply and cause infection; hence, the process causes inactivation of the pathogen.

The percentage reduction of pathogens of any given specific type depends on the integrated product of the UV-C radiant intensity incident on the pathogen and the exposure time. This product is typically called “the applied dose” [symbol] . The energy per unit area incident on a distribution of identical pathogens needed to achieve a reduction in ability to undergo mitosis by a factor of 10 (alternatively, to inactivate 90% of the pathogens in the distribution) is frequently called the LD90. The value of LD90, for a specific pathogen, depends on the wavelength or range of wavelengths. If the applied dose is [symbol] , and [symbol] equals Θ times LD90, then the reduction in the number of viable pathogens as a result of exposure is 10^{-Θ}.

Herein, “sterilization” refers generically to a process for inactivating pathogens. “Technical sanitation” is defined as Θ=4, a reduction in the number of viable pathogens to 10⁻⁴ of the initial number of viable pathogens (alternatively, to inactivate 99.99% of the pathogens in the distribution). It requires application of a radiant energy per unit area equal to 4 times LD90. “Technical sterilization” corresponds to Θ=6 (alternatively, to inactivate 99.9999% of the pathogens in the distribution).

The UV-C output radiation of interest can be excited, for example, by a discharge in a low-pressure argon gas containing mercury vapor; the emitted wavelengths are centered at 253.7 nm. The gas is contained in a discharge tube; the discharge tube wall, typically made of special quartz, is highly transmissive for the wavelengths of interest. As described below, in embodiments of a UV-C source, tubes with a nominal surface emission intensity of about 250 watts/m² at the tube surface can produce a uniform, isotropic intensity in an UV-C exposure chamber of about 500 watts/m². For a wavelength of 253.7 nm, a surface applied dose on the order of 1200 joules/m² is adequate to achieve technical sterilization for all pathogens of interest in a hospital, laboratory, or food-preparation environment. Other sources emitting UV-C radiation in a range (band) of wavelengths near 253.7 nm include xenon lamps and light-emitting diodes.

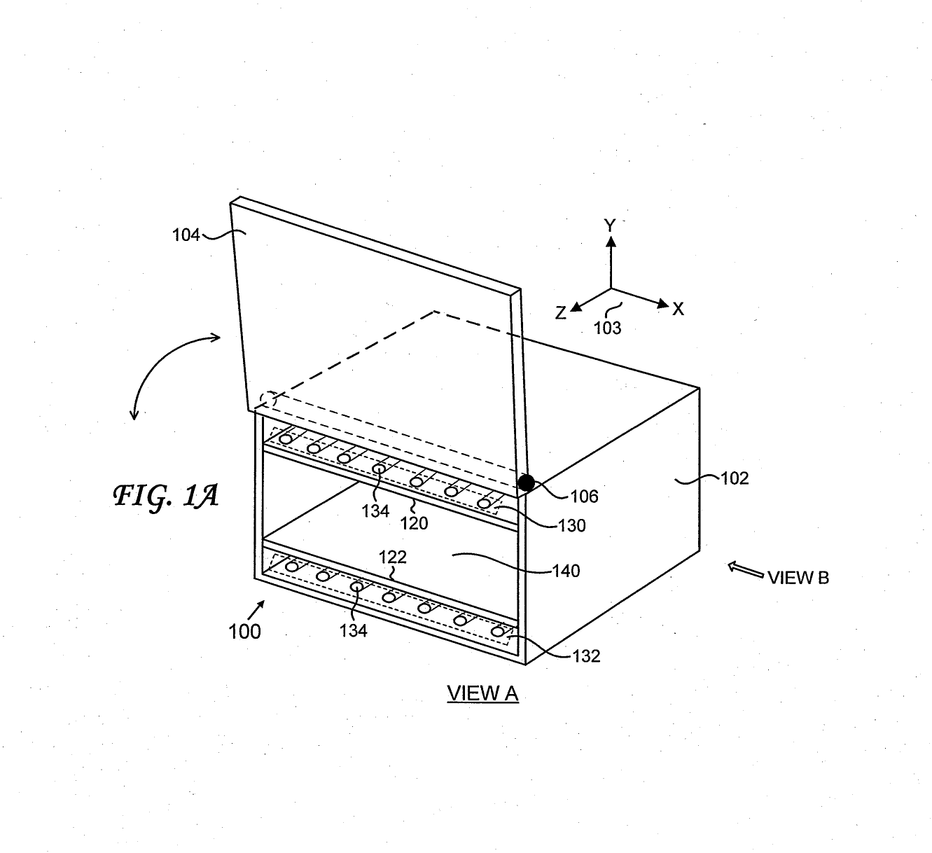

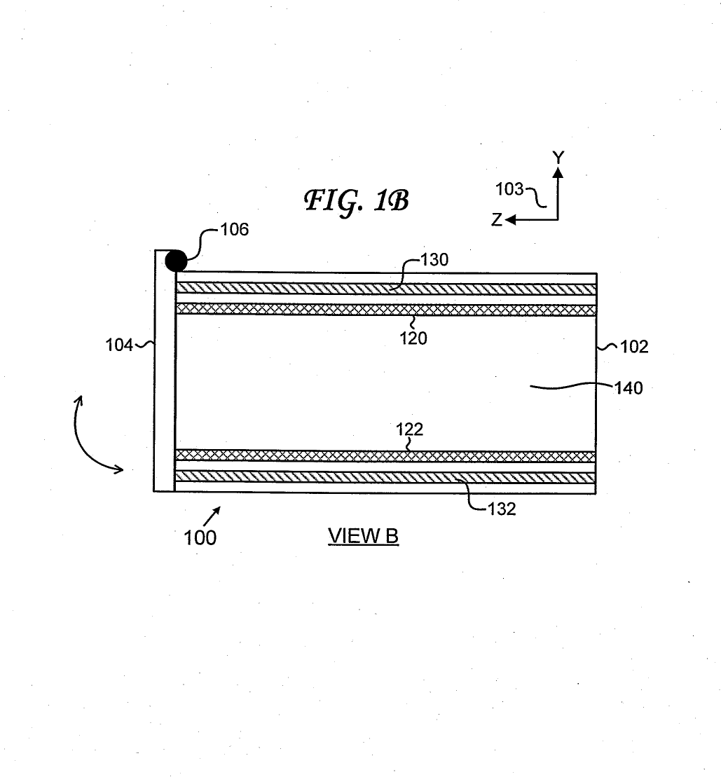

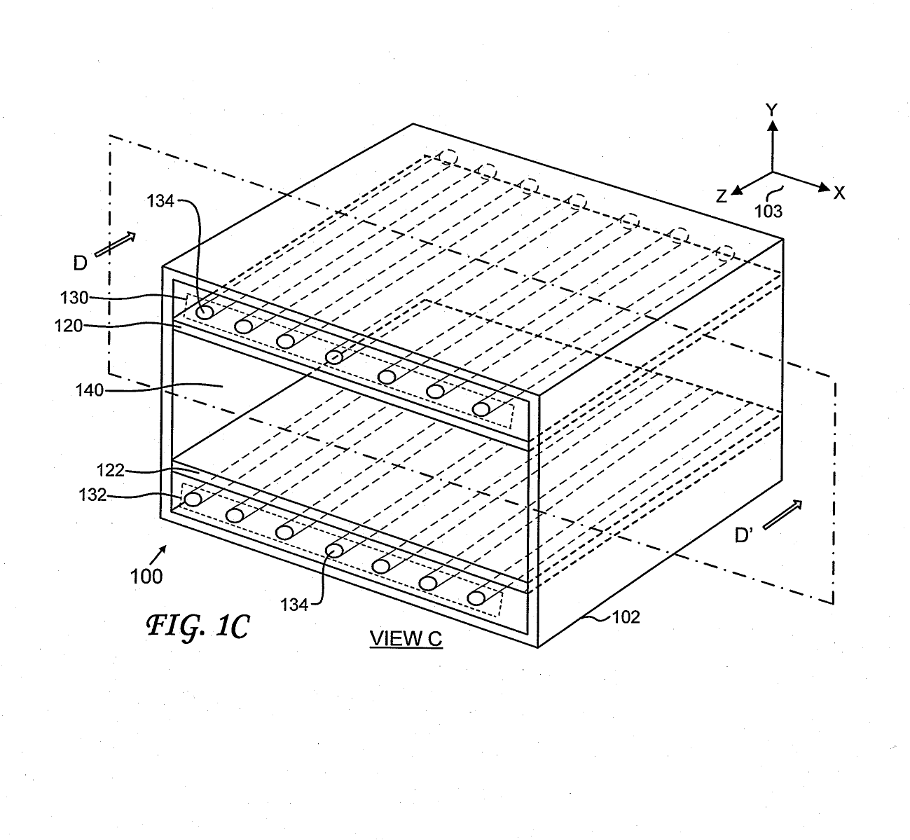

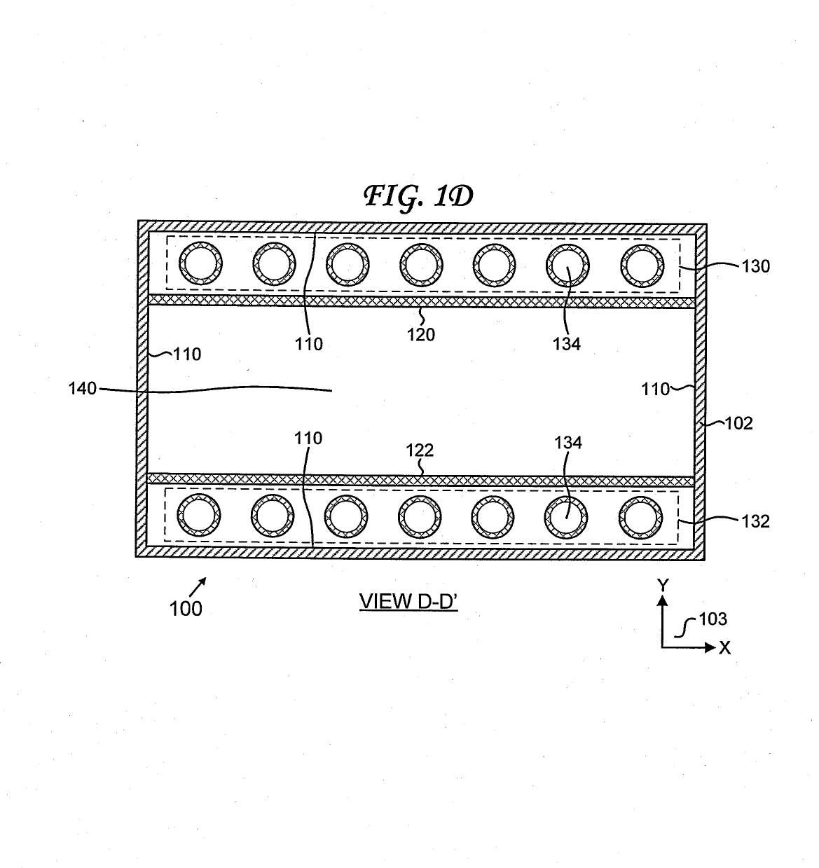

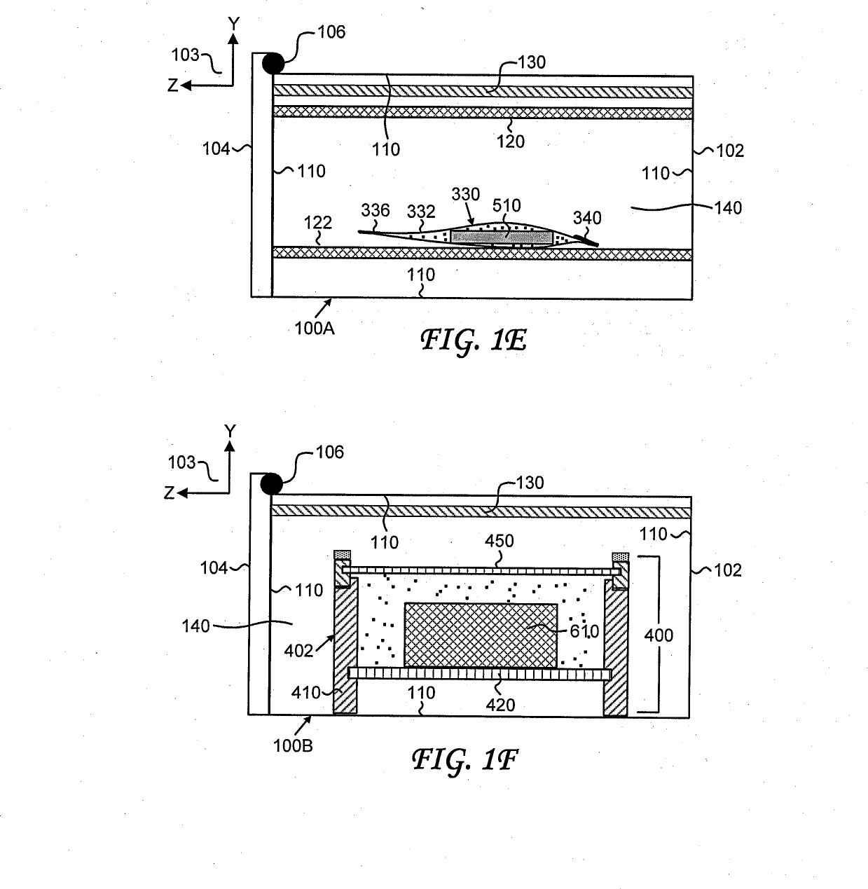

FIG. 1A-FIG. 1D show schematic views of an ultraviolet irradiation device, according to an embodiment of the invention. FIG. 1A shows a perspective view (View A) of the ultraviolet irradiation device 100. A Cartesian X-Y-Z coordinate system 103 is shown for reference. The ultraviolet irradiation device 100 includes the enclosure 102 and the door 104 attached to the enclosure 102 by the hinge 106. The enclosure 102, as shown, is rectangular; in general, other geometries can be used. In FIG. 1A, the door 104 is shown in the open position. Inside the enclosure 102 are two UV-C sources (UV-C source 130 and UV-C source 132) and two partitions (partition 120 and partition 122). More details of the UV-C sources and partitions are described below.

FIG. 1B shows a side view (View B), sighted along the −X axis, of the ultraviolet irradiation device 100. In FIG. 1B, the door 104 is shown in the closed position, and the side wall of the enclosure 102 is not shown. The space bounded by the enclosure 102, the closed door 104, the partition 120, and the partition 122 is referred to as the UV-C exposure chamber 140. Since UV-C radiation can potentially damage skin and eyes, the enclosure 102 and the door 104 are fabricated from materials opaque to UV-C radiation. When the door 104 is closed, a seal, such as a gasket (not shown), prevents leakage of UV-C radiation from the inside of the enclosure 102 to the outside of the enclosure 102. A safety switch (not shown) prevents activation of the UV-C sources when the door 104 is open. To simplify the drawings, electric power supplies and control electronics for the UV-C sources are not shown.

FIG. 1C shows a perspective view (View C) of the ultraviolet irradiation device 100 with the door 104 removed. FIG. 1D shows a cross-sectional view (View D-D′) of the ultraviolet irradiation device 100; the plane of the figure is the X-Y plane.

As shown in FIG. 1A-FIG. 1D, the UV-C source 130, located near the top of the enclosure 102, includes a bank of individual straight UV-C tube sources 134 aligned parallel to the Z-axis; similarly, the UV-C source 132, located near the bottom of the enclosure 102, includes a bank of individual straight UV-C tubes 134 aligned parallel to the Z-axis. The UV-C tubes are not necessarily aligned parallel to the Z-axis; in general, each tube can be independently oriented according to design specifications. Other configurations of UV-C sources can be used. Multiple curvilinear tubes with a “U” shape, or a single curvilinear tube with an “S” shape, for example, can be used as a distributed source.

Refer to FIG. 1D. The interior surface 110 of the enclosure 102 (and the interior surface of the door 104, not shown) is fabricated from a material, or materials, such as aluminum, having a substantially high reflectivity for UV-C radiation. The enclosure 102 (and the door 104, not shown), for example, can be fabricated from sheet aluminum. If the enclosure and the door are fabricated from a material such as plastic, the interior surfaces can be coated with an aluminum film. The interior surfaces reflect UV-C radiation emitted from the UV-C source 130 and the UV-C source 132 back into the UV-C exposure chamber 140 to maximize the UV-C intensity within the UV-C exposure chamber 140.

The partition 120 and the partition 122 are fabricated from material, or materials, substantially transparent to UV-C radiation; suitable materials are discussed below. The partition 120 and the partition 122 can be fabricated from the same material or from different materials. The partition 120 and the partition 122 can have the same thickness (measured along the Y-axis) or different thicknesses. The partition 120 prevents contact with the UV-C source 130, and the partition 122 prevents contact with the UV-C source 132. The partition 122, furthermore, serves as a support shelf on which objects to be sterilized can be placed. The partition 120, therefore, can be thinner and more flexible than the partition 122. As discussed below, other configurations of ultraviolet irradiation devices can be used.

In operation, the door 104 is opened, and the object to be sterilized is placed into the UV-C exposure chamber 140 on the partition 122. The door 104 is then closed, and the UV-C source 130 and the UV-C source 132 are activated for a predetermined exposure time. The door 104 is then opened, and the object is removed.

In an embodiment of the invention, the object to be sterilized is first sealed in a container. The container is fabricated from material, or materials, that are substantially impermeable to pathogens of interest (these pathogens, for example, are specified by applicable medical standards). The seal is also substantially impermeable to the transmission of pathogens. At least a portion of the container is fabricated from material, or materials, that are also substantially transparent to UV-C radiation. Suitable materials are discussed below.

A container can be either flexible or rigid. Herein, a flexible container is referred to as a “pouch” and a rigid container is referred to as a “kit”. A pouch for containing a medical instrument (in particular, a surgical instrument) is referred to as an “instrument pouch”, and a kit for containing a medical instrument is referred to as an “instrument kit”.

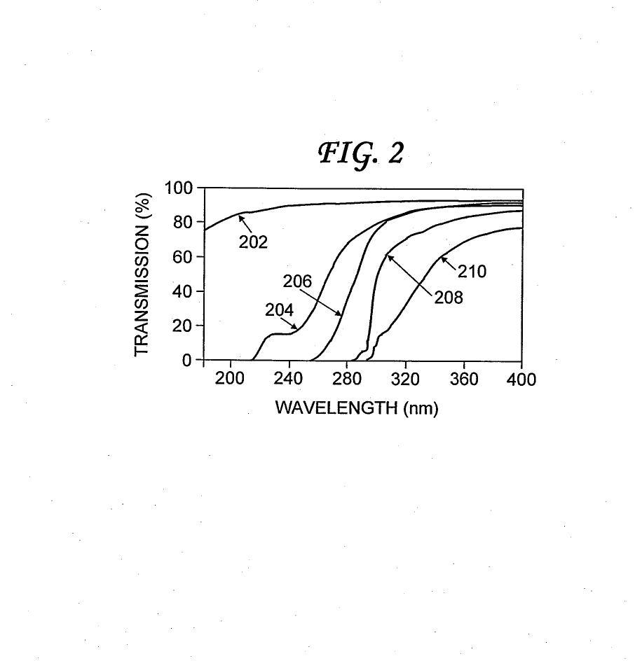

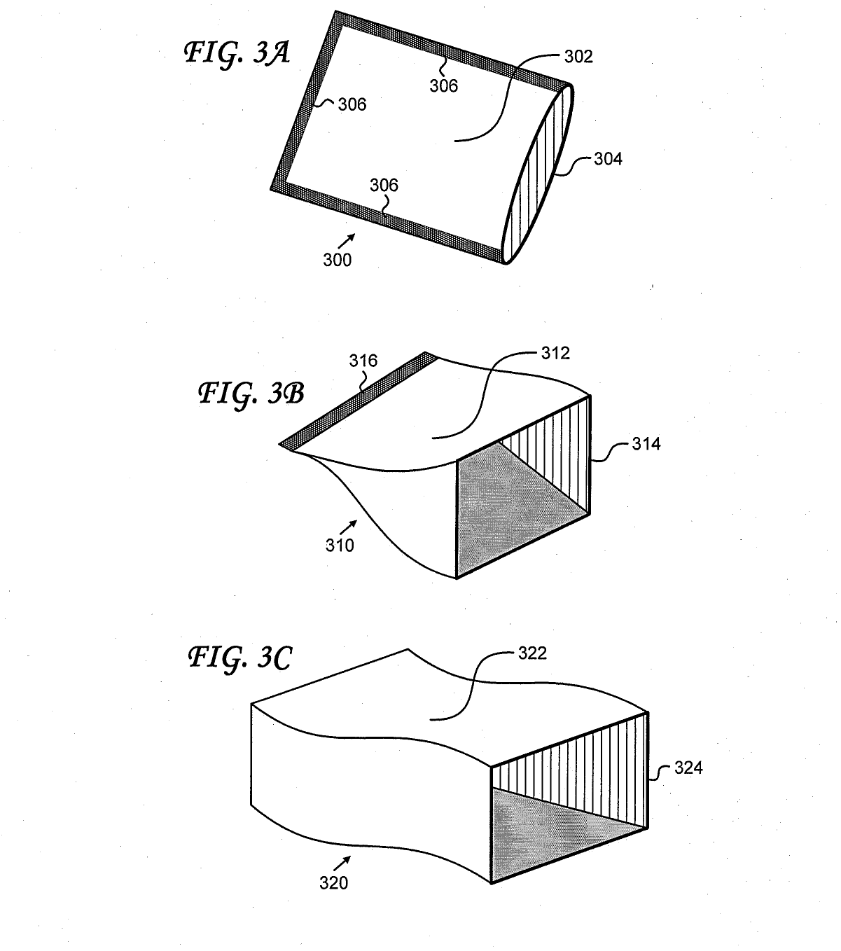

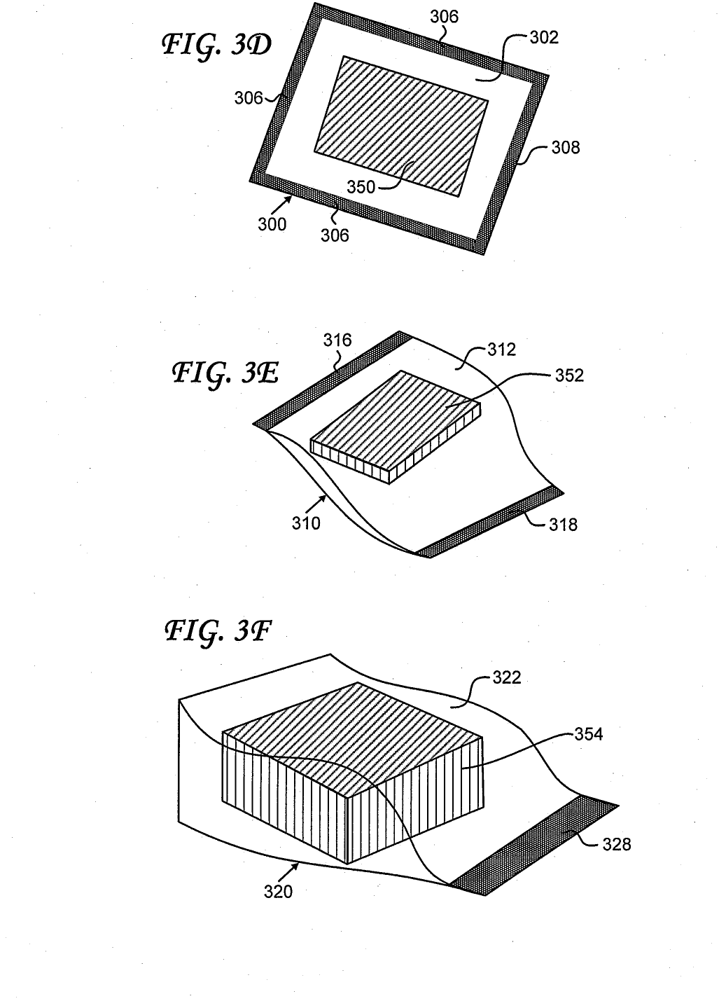

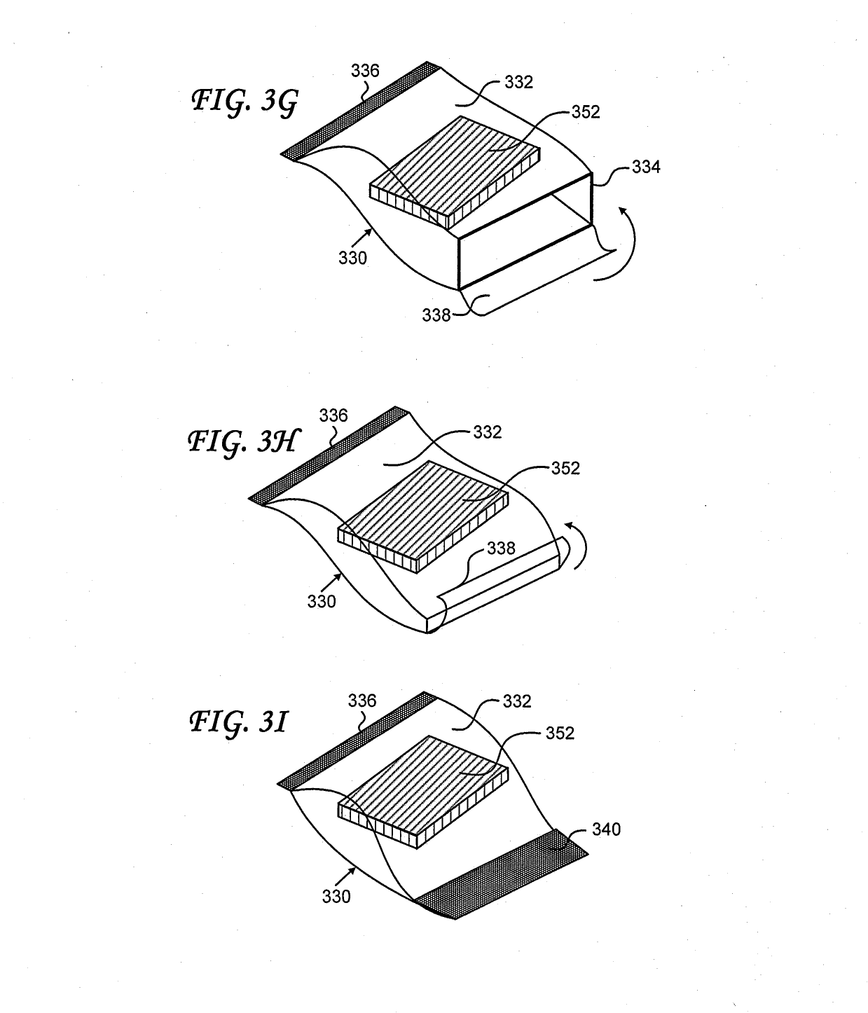

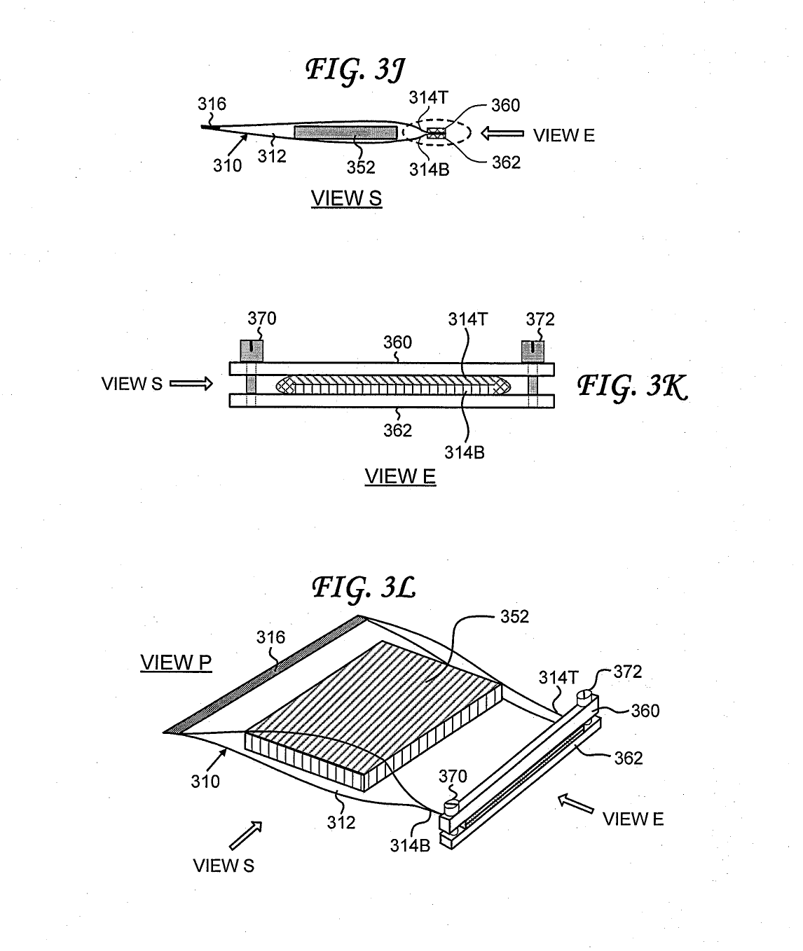

FIG. 3A-FIG. 3C show schematics (perspective views) of three styles of pouches. FIG. 3A shows a flat pouch 300 with a pouch body 302 and an open end 304. Herein, an open end of a pouch is also referred to as a pouch opening. The pouch 300 is fabricated from two thin films or thin sheets sealed along the three seams 306. FIG. 3B shows an intermediate pouch 310 with a pouch body 312 and an open end 314. The pouch 310 is fabricated from a thin film or thin sheet tubular sleeve sealed along the seam 316. FIG. 3C shows a large pouch 320 with a pouch body 322 and an open end 324. The pouch 320 is fabricated from thin film or thin sheet without any seams.

FIG. 3D-FIG. 3F show schematics (perspective views) of objects sealed within pouches. FIG. 3D shows an object 350 inserted into the pouch 300. The pouch 300 is then sealed along the seam 308. FIG. 3E shows an object 352 inserted into the pouch 310. The pouch 310 is then sealed along the seam 318. FIG. 3F shows an object 354 inserted into the pouch 320. The pouch 320 is then sealed along the seam 328.

Other configurations of pouches can be used. FIG. 3G, for example, shows a pouch 330 that is similar to the pouch 310 (FIG. 3B). The pouch 330 has a pouch body 332. The pouch 330 is sealed along the seam 336 and has an open end 334. The pouch 330 also has a flap 338. In FIG. 3G, an object 352 has been inserted into the pouch 330. In FIG. 3H, the flap 338 is wrapped around the open end 334. In FIG. 3I, the flap 338 is then sealed along the seam 340.

Various methods can be used to seal a pouch. Single-use (disposable) pouches, for example, can be sealed with adhesive or by thermal fusion. For multi-use (reusable) pouches, a mechanical seal can be used. FIG. 3J shows a side view (View S) of the object 352 sealed in the pouch 310. The open end 314 is sealed by the clamp bar 360 and the clamp bar 362. The clamp bars can be fabricated from material, or materials, substantially transparent to UV-C radiation. For discussion, the top edge of the opening is referenced as the edge 314T, and the bottom edge of the opening is referenced as the edge 314B.

FIG. 3K shows an end view (View E). Clamping pressure is applied to the clamp bar 360 and the clamp bar 362 to compress the edge 314T and the edge 314B to form a tight seal. The clamping pressure can be applied by various methods. For example, screw clamps, spring clips, or magnets can be attached to both ends of the clamp bars. As shown in FIG. 3K, the clamp bars are fastened together with screws, the screw 370 and the screw 372; the clamp bar 360 has through holes at each end, and the clamp bar 362 has mating threaded holes at each end. Alternatively, the clamp bar 362 can also have through holes at each end, and the screws can be secured with nuts. FIG. 3L shows a perspective view (View P) of the object 352 sealed within the pouch 310. The pouch can be unsealed by loosening the screws and removing the clamp bars. Since the pouch is not torn open or cut open, the pouch can be reused.

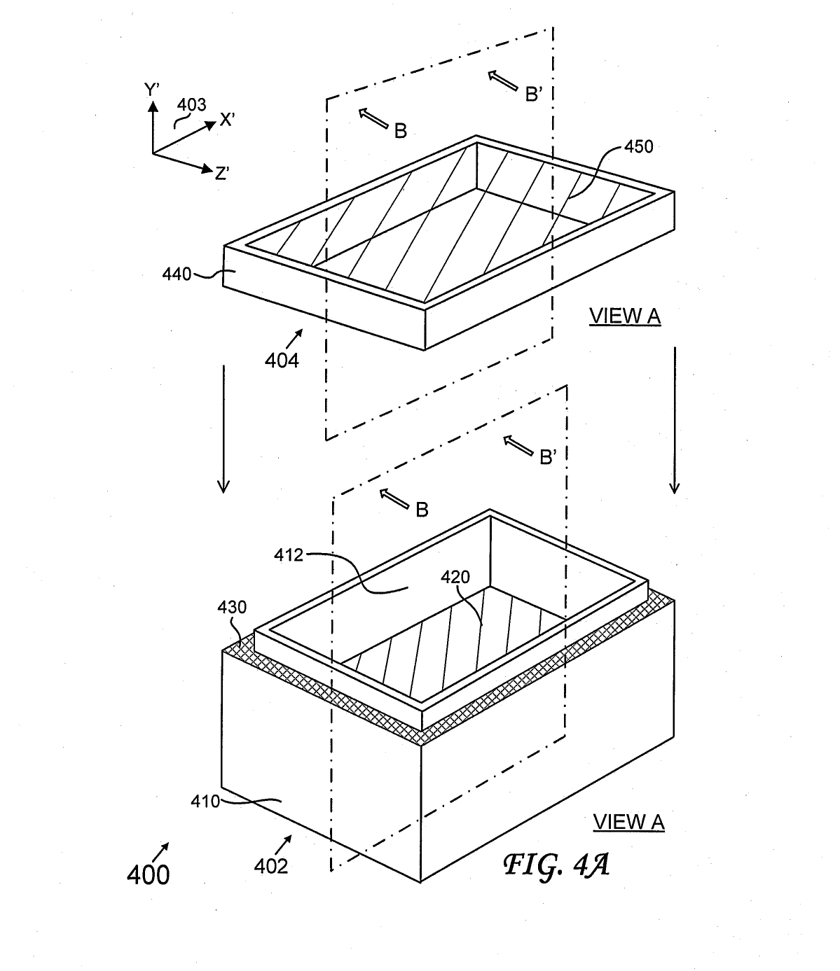

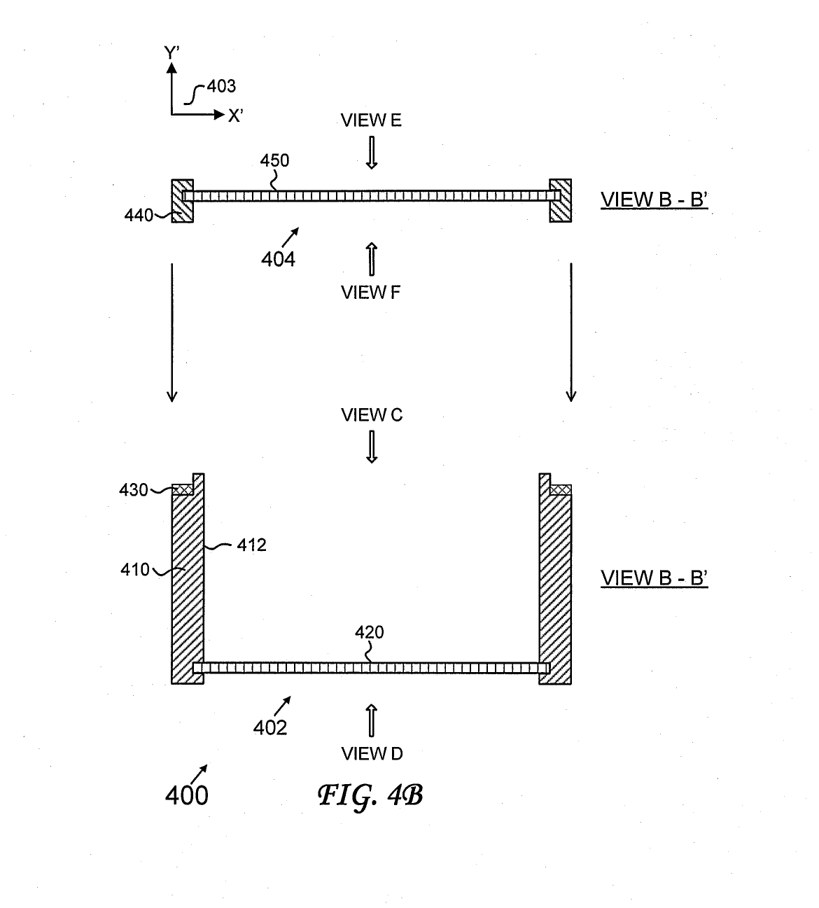

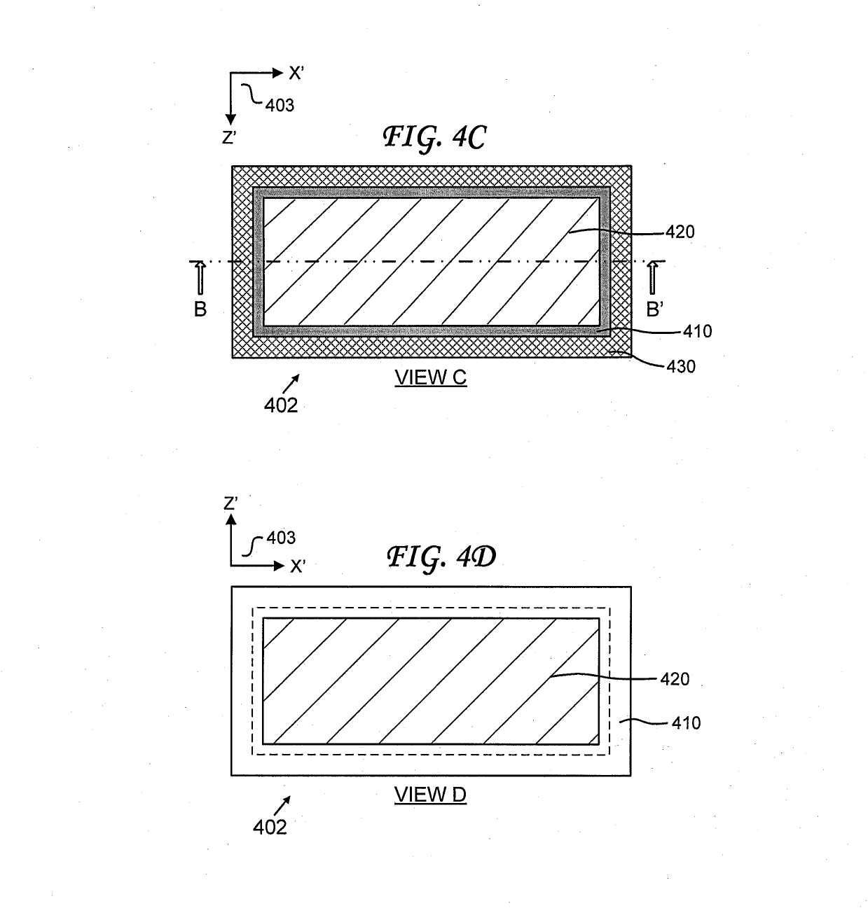

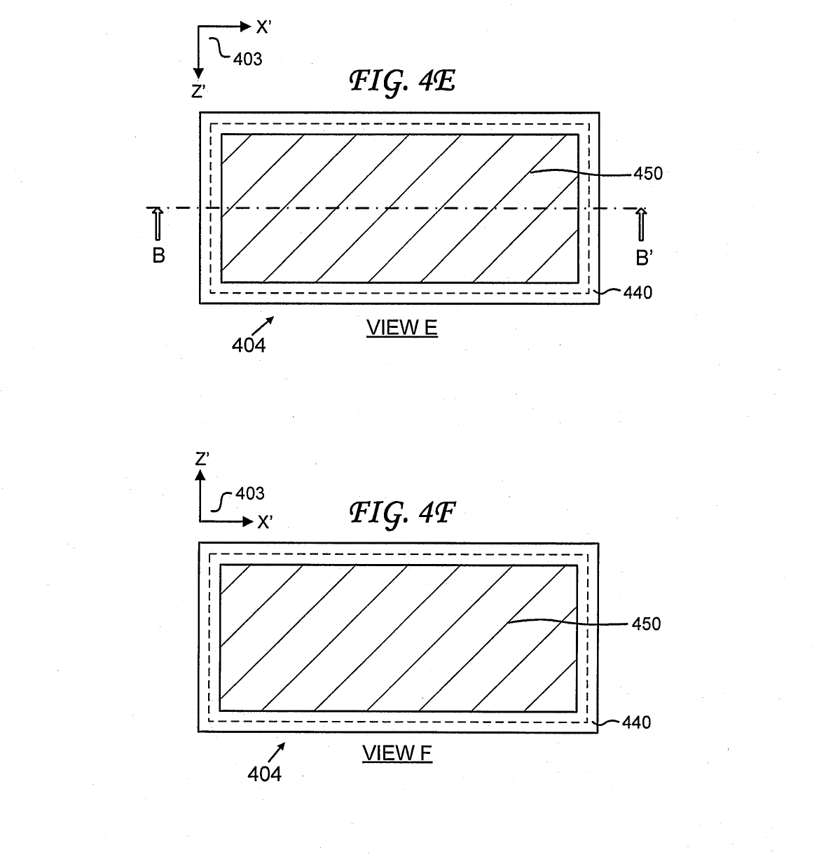

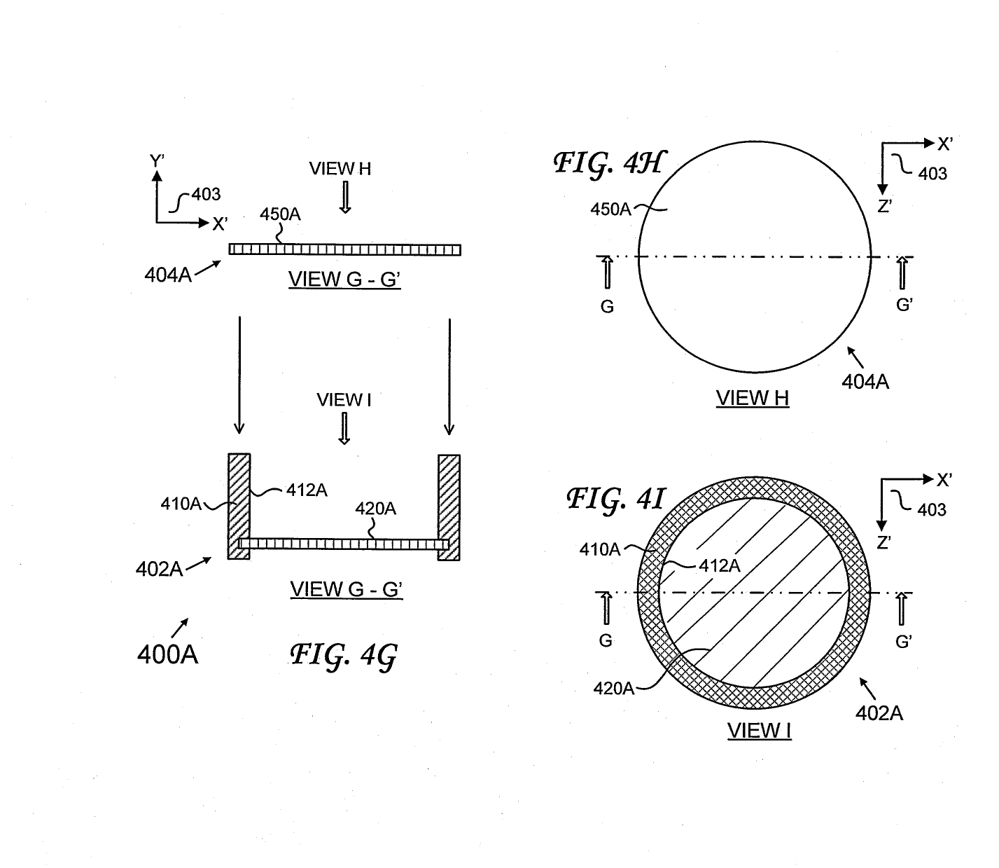

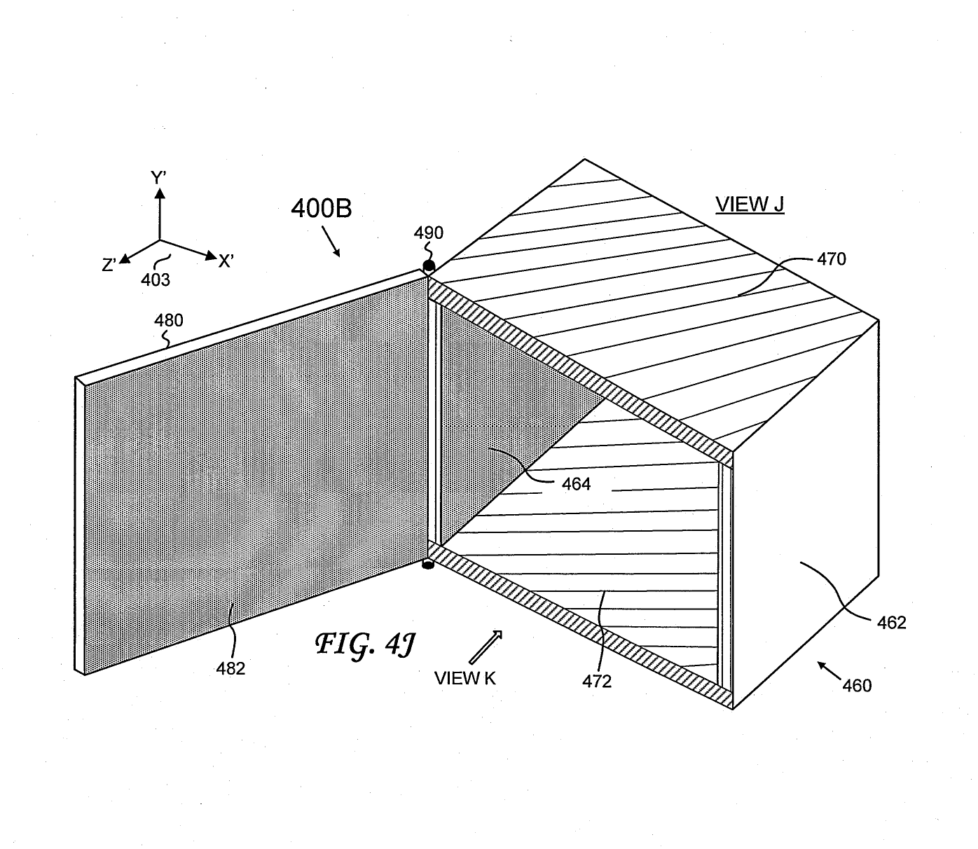

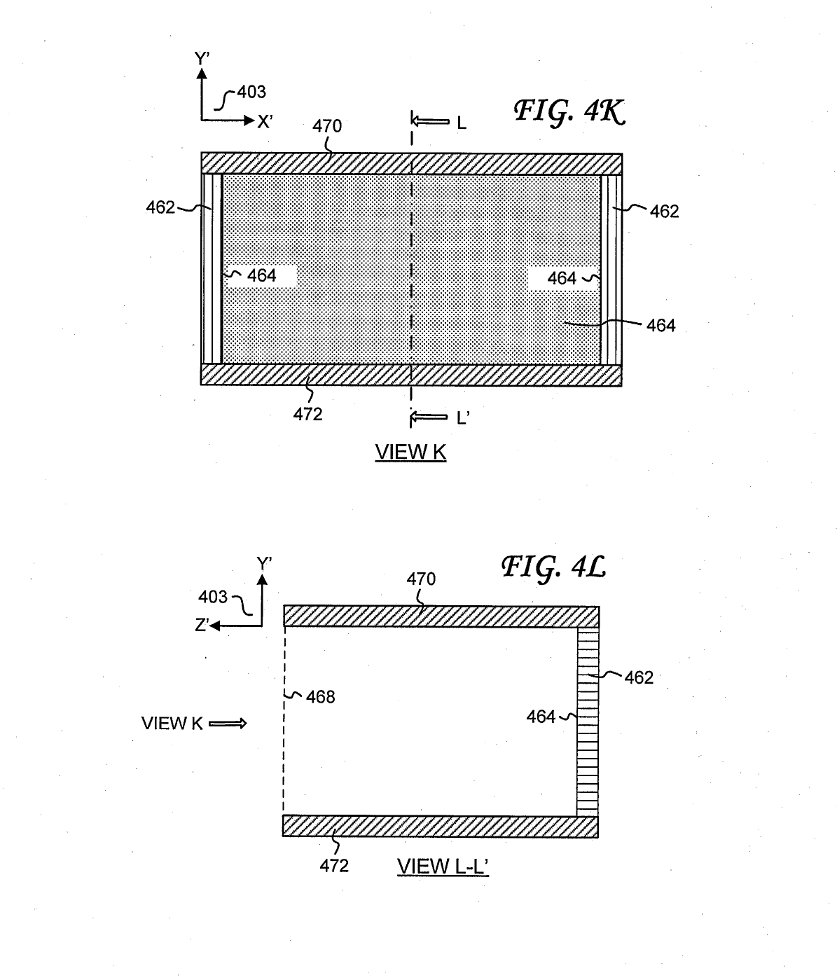

FIG. 4A-FIG. 4F show schematics of a kit, according to an embodiment of the invention. FIG. 4A shows a perspective view (View A) of a kit 400; FIG. 4B shows a cross-sectional view (View B-B′) of the kit 400. The kit 400, as shown, has a rectangular geometry; in general, other geometries can be used. A Cartesian X′-Y′-Z′ coordinate system 403 is shown for reference. When the kit 400 is placed into the ultraviolet irradiation device 100, the Y′-axis of the kit is nominally aligned with the Y-axis of the ultraviolet irradiation device.

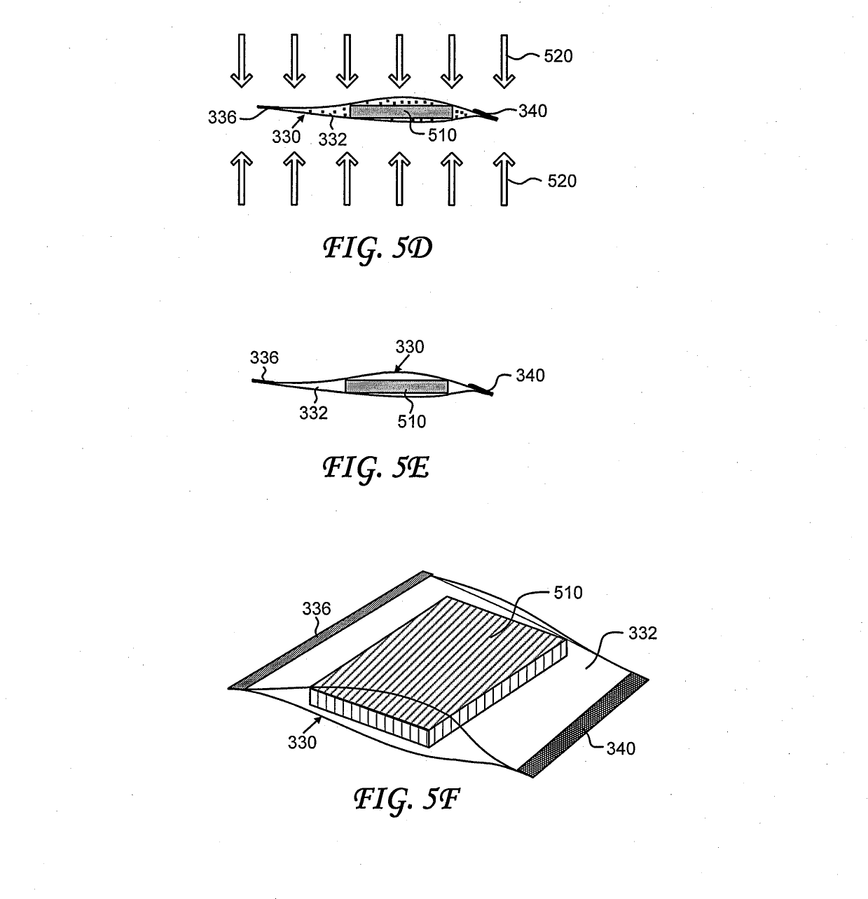

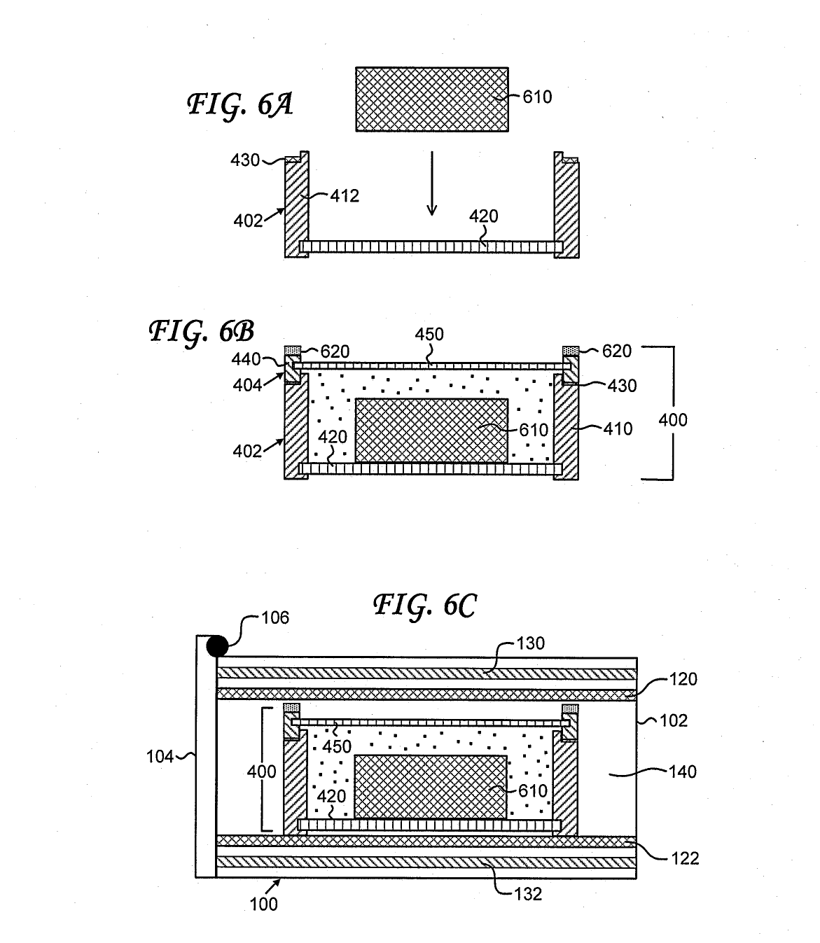

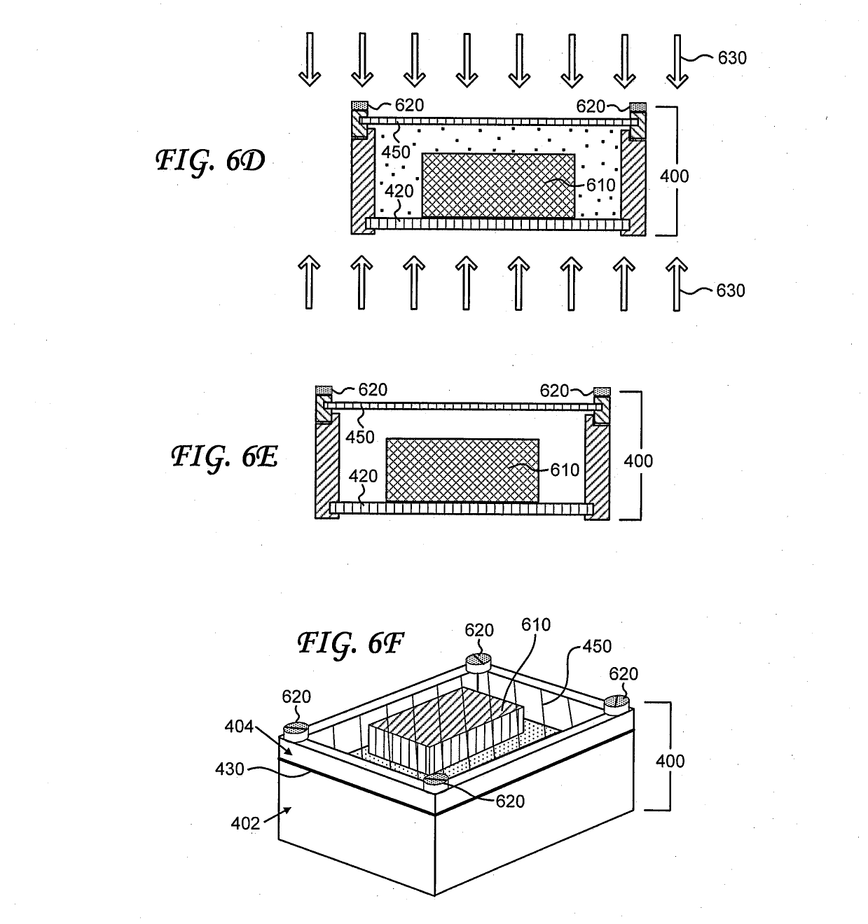

The kit 400 includes the receptacle 402 and the cover 404. In the embodiment shown in FIG. 4A, the cover and the receptacle can be detached; in other embodiments, the cover and the receptacle are coupled together with a hinge. A gasket 430 is placed on a shoulder on the receptacle 402. In other embodiments, a gasket is not used. The cover and the receptacle can be mechanically sealed together. For example, the cover 404 is fitted onto the receptacle 402, and the cover 404 and the receptacle 402 are clamped together to form a seal substantially impermeable to the transmission of specified pathogens. Various clamping mechanisms, such as screw clamps, spring bands, and magnets can be used. The cover 440 can also be fastened to the receptacle 402 with screws (see FIG. 6F below).

The receptacle 402 includes the side wall 410 and the bottom plate 420. As shown, the side wall is flat and oriented orthogonal to the bottom plate. In general, the side wall can be flat or curved and can be oriented non-parallel to the bottom plate. The bottom plate 420 is fabricated from material, or materials, substantially transparent to UV-C radiation. In some embodiments, the side wall is also fabricated from material, or materials, substantially transparent to UV-C radiation. In other embodiments, the interior surface 412 (in part or in entirety) of the side wall is fabricated from a material, such as aluminum, having substantially high reflectivity for UV-C radiation. For example, the side wall 410 can be fabricated from sheet aluminum; or the side wall 410 can be fabricated from plastic, and the interior surface 412 can be coated with aluminum film.

FIG. 4C shows a top view (View C), sighted along the −Y′ axis, of the receptacle 402; FIG. 4D shows a bottom view (View D), sighted along the +Y axis, of the receptacle 402. In some embodiments, the receptacle 402 is fabricated from several pieces sealed together. In other embodiments, the receptacle 402 is fabricated as a single piece. Herein, various portions of a receptacle (or any item) fabricated as a single piece are also considered to be “sealed together”.

The cover 404 includes the side wall 440 and the top plate 450. As shown, the side wall is flat and oriented orthogonal to the top plate. In general, the side wall can be flat or curved and can be oriented non-parallel to the top plate. Both the side wall 440 and the top plate 450 are fabricated from material, or materials, substantially transparent to UV-C radiation. FIG. 4E shows a top view (View E), sighted along the −Y′ axis, of the cover 404; FIG. 4F shows a bottom view (View F), sighted along the +Y axis, of the cover 404. In some embodiments, the cover 404 is fabricated from several pieces sealed together. In other embodiments, the cover 404 is fabricated as a single piece.

FIG. 4G-FIG. 4I show schematics of a second kit, according to an embodiment of the invention. FIG. 4G shows a cross-sectional view (View G-G′) of the kit 400A, which includes the receptacle 402A and the cover 404A. When the kit 400A is placed into the ultraviolet irradiation device 100, the Y′-axis of the kit is nominally aligned with the Y-axis of the ultraviolet irradiation device. In the embodiment shown in FIG. 4G, the cover and the receptacle can be detached; in other embodiments, the cover and the receptacle are coupled together with a hinge. The kit 400A has a cylindrical geometry. FIG. 4H shows a top view (View H), sighted along the−Y′ axis, of the cover 404A; FIG. 4I shows a top view (View I), sighted along the −Y′ axis, of the receptacle 402A.

The cover 450A is a circular plate fabricated from material, or materials, substantially transparent to UV-C radiation. In the embodiment shown in FIG. 4G, the cover 450A has no side wall; in other embodiments, the cover has a side wall. The receptacle 402A includes the tubular side wall 410A and the circular bottom plate 420A. The bottom plate 402A is fabricated from material, or materials, substantially transparent to UV-C radiation.

In some embodiments, the side wall is also fabricated from material, or materials, substantially transparent to UV-C radiation. In other embodiments, the interior surface 412A (in part or in entirety) of the side wall is fabricated from a material, such as aluminum, having a substantially high reflectivity for UV-C radiation. For example, the side wall 410A can be fabricated from sheet aluminum; or the side wall 410A can be fabricated from plastic, and the interior surface 412A can be coated with aluminum film. In some embodiments, the receptacle 402A is fabricated from several pieces sealed together. In other embodiments, the receptacle 402A is fabricated as a single piece.

The cover and the receptacle can be mechanically sealed together. In the embodiment shown in FIG. 4G, no gasket is placed between the cover and the receptacle; in other embodiments, a gasket is used. The cover 404A and the receptacle 402A can be clamped together or screwed together.

FIG. 4J-FIG. 4L show schematics of a third kit, according to an embodiment of the invention. FIG. 4J shows a perspective view (View J) of a kit 400B, which includes the receptacle 460 and the cover 480. When the kit 400B is placed into the ultraviolet irradiation device 100, the Y′-axis of the kit is nominally aligned with the Y-axis of the ultraviolet irradiation device. The kit 400B, as shown, has a rectangular geometry; in general, other geometries can be used. In the embodiment shown in FIG. 4J, the cover and the receptacle are coupled together by the hinge 490; in other embodiments, the cover and the receptacle can be detached.

FIG. 4K shows a front view (View K), sighted along the −Z′ axis, of the receptacle 460. FIG. 4L shows a cross-sectional view (View L-L′) of the receptacle 460. The receptacle 460 includes a top plate 470, a bottom plate 472, and a side wall 462. As shown, the side wall is flat and oriented orthogonal to the top and bottom plates. In general, the side wall can be flat or curved and can be oriented non-parallel to the top and bottom plates. The opening 468 is opposite to a portion of the side wall.

The top plate 470 and the bottom plate 472 are fabricated from material, or materials, substantially transparent to UV-C radiation; the top plate 470 and the bottom plate 472 can be fabricated from the same material or from different materials. In some embodiments, the side wall is also fabricated from material, or materials, substantially transparent to UV-C radiation. In other embodiments, the interior surface 464 (in part or in entirety) of the side wall is fabricated from a material, such as aluminum, having a substantially high reflectivity for UV-C radiation. For example, the side wall 462 can be fabricated from sheet aluminum; or the side wall 462 can be fabricated from plastic, and the interior surface 464 can be coated with aluminum film.

Refer back to FIG. 4J. The cover 480 is a rectangular plate. In some embodiments, the cover 480 is fabricated from material, or materials, substantially transparent to UV-C radiation. In other embodiments, the interior surface 482 (in part or in entirety) of the cover is fabricated from a material, such as aluminum, having a substantially high reflectivity for UV-C radiation. For example, the cover 480 can be fabricated from sheet aluminum; or the cover 480 can be fabricated from plastic, and the interior surface 482 can be coated with aluminum film.

The cover and the receptacle can be mechanically sealed together. In the embodiment shown in FIG. 4J, no gasket is placed between the cover and the receptacle; in other embodiments, a gasket is used. The cover 480 and the receptacle 460 can be clamped together or screwed together.

FIG. 2 shows light transmission (%) as a function of wavelength (nm) for 2-mm thick samples of various materials. Plot 202 shows the results for quartz glass (fused silica). Plot 204 shows the results for TOPAS 8007X10 (see below). Plot 206 shows the results for PMMA [poly(methyl methacrylate)]. Plot 208 shows the results for PS (polystyrene). Plot 210 shows the results for PC (polycarbonate).

Quartz, due to its high UV-C transmission, is advantageous for the envelope of a UV-C lamp emitting at a wavelength λ=253.7 nm. Borosilicate glass, not shown, can have >80% transmission at λ=253.7 nm for a thickness of 2 mm and can also be used for UV-C tubes (see, for example, U.S. Pat. No. 5,547,904 and U.S. Pat. No. 5,610,108). For quartz, the bulk absorption loss is negligible, and the fact that the transmission factor is not quite 100% results from reflection loss at the two quartz-air interfaces.

Reflection does not actually result in an intensity loss in a UV-C tube, and also not in the ultraviolet irradiation process described herein, because the reflected photons are not lost. Rather, a reflected λ=253.7 nm photon moving back into the tube is absorbed by a ground state mercury atom (because the lower level of the radiating transition is the ground state). The mercury atom is consequently excited to the λ=253.7 nm radiating level which then transitions back to the ground state as it re-emits the photon. The reflected photon is not lost. Hence, these curves underestimate the effective transmission factor. In embodiments of a UV-C source, tubes with a nominal surface emission intensity of about 250 watts/m² at the tube surface produce a uniform, isotropic intensity in the UV-C exposure chamber of about 500 watts/m².

Quartz and low-loss borosilicate glass can also be used for the partition 120 and the partition 122 in the ultraviolet irradiation device 100 (FIG. 1D); the bottom plate 420, the top plate 450, the side wall 410, and the side wall 440 in the kit 400 (FIG. 4B); the bottom plate 420A, the cover 450A, and the side wall 410A in the kit 400A (FIG. 4G); the top plate 470, the bottom plate 472, the side walls (462, 464, and 466), and the cover 480 in the kit 400B (FIG. 4J); and the clamp bar 360 and the clamp bar 362 for mechanical seals (FIG. 3J-FIG. 3L).

Quartz and low-loss borosilicate glass are rigid materials and are therefore not suitable materials for a pouch. A pouch could be made of PMMA, but its transmission loss would be high. An advantageous choice of pouch material is TOPAS Cyclic Olefin Copolymer (TOPAS COC), or variations thereof, which has good physical properties and is used for packaging medical devices, medicines, and food. (For information on TOPAS COC, see “TOPAS Cyclic Olefin Copolymer (COC)”, pp. 1-20, Topas Advanced Polymers GmbH, Frankfurt, Germany, and Topas Advanced Polymers, Inc., Florence, Ky., USA, March 2006.) In particular, TOPAS COC 8007X10 has a UV-C transmission at τ=253.7 nm of about 20% in a 2-mm thickness as shown in plot 204. The percent transmission factor at τ=253.7 nm, \check{T} (λ=253.7 nm) in %, as a function of the thickness of the TOPAS COC 8007X10, τ, in mm, is given by the Beer-Lambert Law, in which the parameter 0.8045 is determined from the plot 204 with τ=2 mm: \check{T} (λ=253.7 nm)=100 exp(−0.8045 τ). From the Beer-Lambert Law, the following values are obtained (1 mil=0.001 inch):
For τ=2 mm (50.8 mils), \check{T}≈20.0%
For τ=1 mm (25.4 mils), \check{T}≈44.7
For τ=0.1575 mm (4 mils), \check{T}≈88.1%.

Reflection loss will reduce the 4-mil value by about 8% to about \check{T}≈80%, but does not increase the actual intensity loss in the application of interest (pouch for UV-C irradiation). In a direct transmission measurement at λ=253.7 nm, for a 4-mil thick film of TOPAS COC 8007X10, it was determined that \check{T}=90% with reflection loss. A disadvantage of TOPAS COC 8007X10 is that it degrades under UV-C irradiation; that is, the transmission loss increases with repeated UV-C exposure. The relatively low cost of TOPAS COC 8007X10, however, makes it well suited for single-use, disposable pouches (FIG. 3A-FIG. 3I). As discussed above, a surface applied dose on the order of 1200 joules/m² is adequate to achieve technical sterilization for all pathogens of interest in a hospital, laboratory, or food-preparation environment. Assuming that the thin film of the pouch transmits at least 80% of the incident UV-C radiation, the ultraviolet irradiation device 100 (FIG. 1A) can achieve technical sterilization of surfaces in about 3 sec.

Another advantageous material for containers is Teflon AF, a fluoropolymer. (For information on Teflon AF, see M. K. Yang et al., “Optical properties of Teflon AF amorphous fluoropolymers”, J. Micro/Nanolith. MEMS MOEMS 7(3), 033010 (July-September 2008), pp. 033010-1 to 033010-9, and references cited therein.) Teflon AF is strong, durable, and commercially available in pliable thin films and rigid sheets. It has optical transmission characteristics similar to that of UV-C transparent quartz. The transmission factor is limited only by reflection and not by loss. Moreover, Teflon AF does not degrade when exposed to UV-C radiation. Although it can be used for single-use, disposable pouches, it is substantially more expensive than TOPAS COC. Pliable thin films of Teflon AF, therefore, are well suited for multi-use (reusable) pouches (see FIG. J-FIG. 3L). Rigid sheets of Teflon AF, furthermore, can be used for the partition 120 and the partition 122 in the ultraviolet irradiation device 100 (FIG. 1D); the bottom plate 420, the top plate 450, the side wall 410, and the side wall 440 in the kit 400 (FIG. 4B); the bottom plate 420A, the cover 450A, and the side wall 410A in the kit 400A (FIG. 4G); the top plate 470, the bottom plate 472, the side walls (462, 464, and 466), and the cover 480 in the kit 400B (FIG. 4J); and the clamp bar 360 and the clamp bar 362 for mechanical seals (FIG. 3J-FIG. 3L).

In practice, a material is substantially transparent to UV-C radiation according to the following criteria. Assume that a sheet or film of the material with a specified thickness is placed between a UV-C source and a surface contaminated with pathogens. Assume that the UV-C intensity incident on the sheet or film has a specified value. The UV-C intensity incident on the surface contaminated with pathogens depends on the UV-C intensity incident on the sheet or film and the transmission loss in the sheet or film. The material is substantially transparent to UV-C radiation if a specified portion of the pathogens disposed on the surface of the object is inactivated within a specified exposure time. For example, for technical sterilization, with a UV-C wavelength of about 253.7 nm and a UV-C intensity incident on the sheet or film of about 500 watts/m², 99.9999% of pathogens of interest can be inactivated within an exposure time on the order of seconds. For example, C. difficile, with an LD90 value of 200 joules/m², requires an exposure time of about 2.4 sec for technical sterilization; and anthrax, with an LD90 value of 750 joules/m², requires an exposure time of about 9 sec.

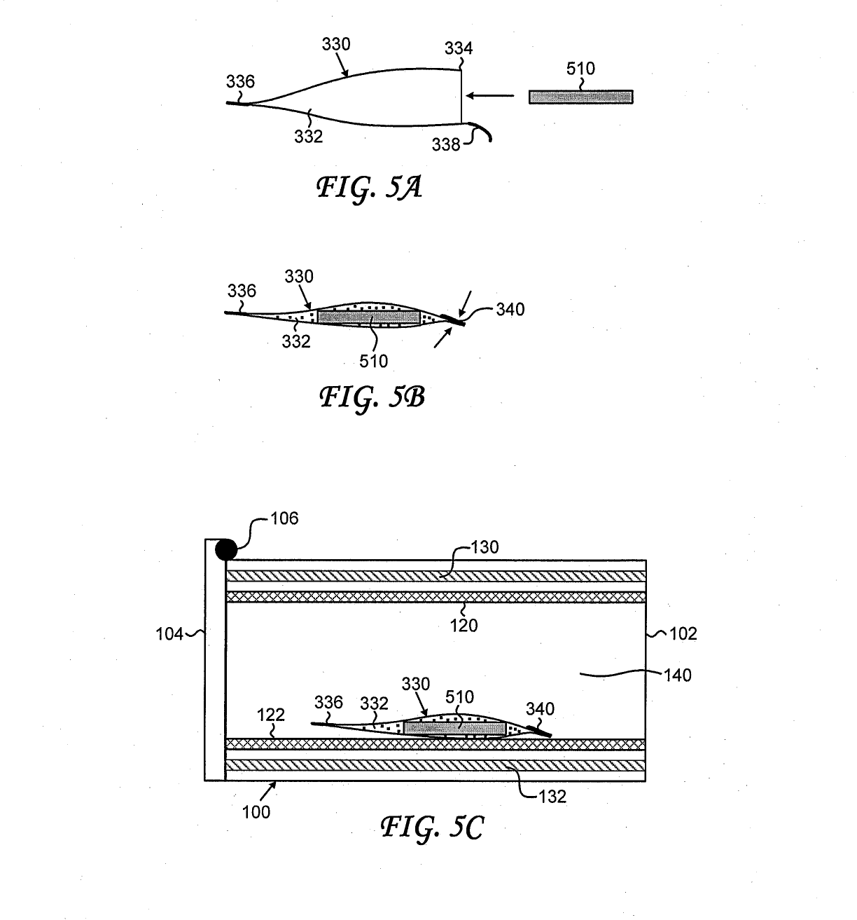

FIG. 5A-FIG. 5E schematically illustrate a method, according to an embodiment of the invention, for sterilizing an object sealed in a pouch. In step 1 (FIG. 5A), the object 510 is inserted into the pouch 330. In step 2 (FIG. 5B), the pouch 330 is sealed. The specks within the pouch represent active pathogens. In step 3 (FIG. 5C), the pouch 330 and the object 510 are placed into the UV-C exposure chamber 140 of the ultraviolet irradiation device 100. In step 4 (FIG. 5D), the pouch 330 and the object 510 are irradiated with the UV-C radiation 520. In step 5 (FIG. 5E), the pouch 330 and the object 510 are removed from the ultraviolet irradiation device 100. The absence of specks within the pouch 330 indicates that the pathogens have been inactivated. FIG. 5F shows a perspective view of the sterilized pouch and object.

FIG. 6A-FIG. 6E schematically illustrate a method, according to an embodiment of the invention, for sterilizing an object sealed in a kit. In step 1 (FIG. 6A), the object 610 is inserted into the receptacle 402. In step 2 (FIG. 6B), the cover 404 is clamped onto the receptacle 402 with the clamping screws 620 to form the sealed kit 400. The specks within the kit represent active pathogens. In step 3 (FIG. 6C), the kit 400 and the object 610 are placed into the UV-C exposure chamber 140 of the ultraviolet irradiation device 100. In step 4 (FIG. 6D), the kit 400 and the object 610 are irradiated with the UV-C radiation 630. In step 5 (FIG. 6E), the kit 400 and the object 610 are removed from the ultraviolet irradiation device 100. The absence of specks within the kit 400 indicates that the pathogens have been inactivated. FIG. 6F shows a perspective view of the sterilized kit and object.

FIG. 1A showed an ultraviolet irradiation device 100 with a UV-C source and a partition near the top of the enclosure and a UV-C source and a partition near the bottom of the enclosure. Depending on the application, other configurations of ultraviolet irradiation devices can be used.

In FIG. 1E, for example, the ultraviolet irradiation device 100A does not have a bottom UV-C source. UV-C radiation emitted from the top UV-C source 130 is reflected by the interior surfaces 110. The pouch 330 containing the object 510 is placed on the bottom partition 122. The bottom of the pouch 330 and the bottom of the object 510 are irradiated by UV-C radiation reflected from the interior surfaces 110.

In FIG. 1F, for example, the ultraviolet irradiation device 100B has a top UV-C source 130, no bottom UV-C source, and no partitions. The kit 400 containing the object 610 is placed on the bottom interior surface 110. The side walls 410 of the receptacle 402 are fabricated from material, or materials, substantially transparent to UV-C radiation (the interior surfaces of the side walls 410 within the sealed region of the receptacle 402 can be coated with aluminum film). The bottom plate 420 is sufficiently raised from the bottom interior surface 110 such that the bottom plate 420 and the bottom of the object 610 are irradiated by UV-C radiation emitted from the UV-C source 130 and reflected by the interior surfaces 110.

In an embodiment of the invention, radio-frequency identification (RFID) tags are attached to pouches, kits, and ultraviolet irradiation devices. A control system can determine whether a specific pouch or a specific kit is compatible with a specific ultraviolet irradiation device. If the control system determines that a specific pouch or specific kit is not compatible with a specific ultraviolet irradiation device, it can send a notification (for example, via an audible alarm or via a flashing indicator) to the operator; the control system can also prevent the UV-C sources from being activated.

All current sterilizers require periodic testing with a biological indicator (BI) to certify that the sterilization process actually achieves sterilization of a particular test endospore. A conventional biological indicator includes a vial containing test pathogens. The biological indicator is processed along with the object to be sterilized. Full technical sterilization certification with a biological indicator requires approximately 24 hours or more; hence, it is not done with every load. The systems do require a process indicator (PI) with each load. The process indicator indicates that the technical sterilization process was fully executed but does not guarantee that technical sterilization was actually achieved.

In an embodiment of the invention, a biological indicator for UV-C sterilization includes a closed vial containing test pathogens; the closed vial is fabricated from material, or materials, substantially transparent to UV-C radiation. The closed vial is placed in the UV-C exposure chamber along with the pouch or kit containing the object to be sterilized.

In an embodiment of the invention, a process indicator is used to monitor operation of the ultraviolet irradiation device. A substrate coated with special phosphors is placed within the pouch or kit, along with the object to be sterilized. The substrate, for example, can be a small quartz disc. When the phosphors are irradiated with UV-C radiation, the phosphors produce long-lived, narrow-band, visible light photo-luminescence. Suitable phosphors include those used in conventional fluorescent lamps. A typical “cool white” fluorescent lamp utilizes two rare earth doped phosphors, Tb³⁺, Ce³⁺: LaPO₄, for green and blue emission. The intensity of the photo-luminescence is a measure of the total UV-C dose delivered to the interior of the pouch or kit. A small narrow-band spectrophotometer, such as a photodiode, can be mounted inside the ultraviolet irradiation device. The spectrophotometer measures the photo-luminescence emitted from within the sealed pouch or kit and indicates that a specified UV-C dose has been delivered.

Pathogens are not inactivated on a surface or in a space that is not exposed to UV-C radiation. Surfaces in contact about a pivot and a space within a cavity are typically shielded from UV-C radiation. In some embodiments of the invention, portions of an object are fabricated from material, or materials, substantially transparent to UV-C radiation such that all surfaces (external and internal) on the object and all spaces within the object are exposed to UV-C radiation. In other embodiments of the invention, an object is fabricated entirely of material, or materials, substantially transparent to UV-C radiation.

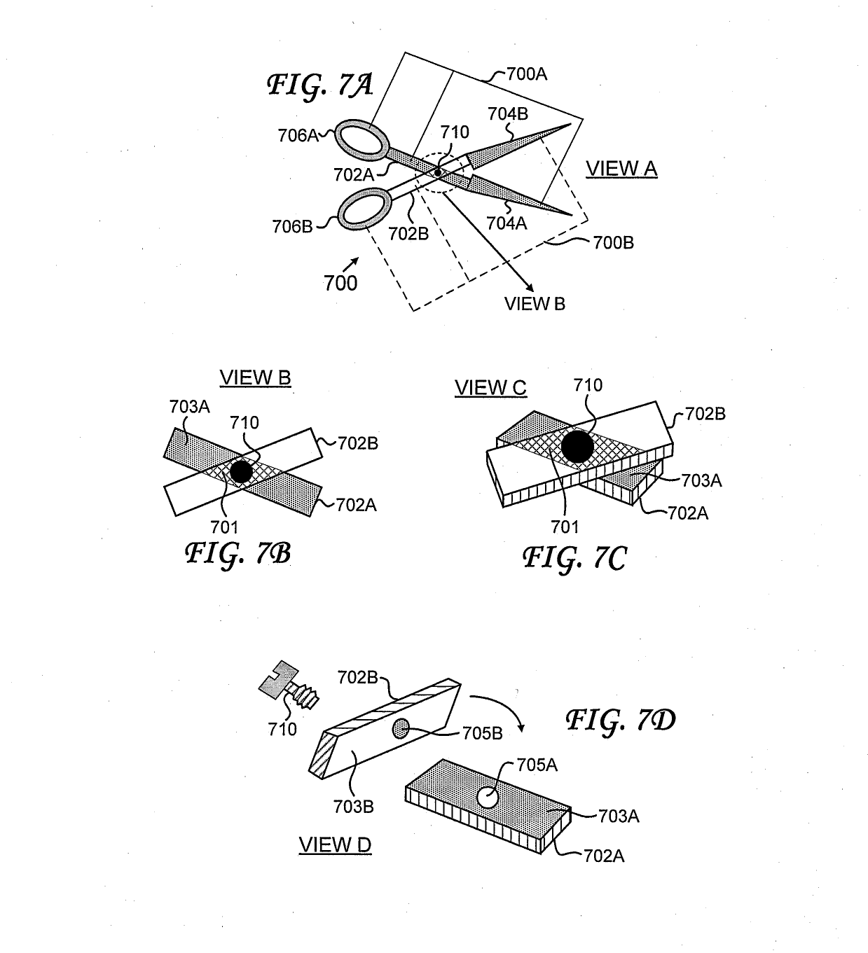

FIG. 7A-FIG. 7D show an example of an object with a pivot joint. Examples of medical instruments with a pivot joint include surgical scissors and surgical forceps. As shown in the plan view (View A) of FIG. 7A, the pair of surgical scissors 700 includes the component 700A and the component 700B operatively coupled by the pivot 710. The component 700A includes the arm 702A, the blade 704A, and the handle 706A. The component 700B includes the arm 702B, the blade 704B, and the handle 706B. Surgical scissors are typically fabricated entirely of stainless steel, which is opaque to UV-C radiation.

FIG. 7B shows a magnified plan view (View B) of a portion of the surgical scissors 700 in a region about the pivot 710. FIG. 7C shows a perspective view (View C) of the same region. FIG. 7D shows a perspective view (View D) of the same region after the surgical scissors 700 has been disassembled and the pivot 710 has been removed.

Refer to FIG. 7D. The arm 702A has a top surface 703A and a hole 705A. The arm 702B has a bottom surface 703B and a hole 705B. When the surgical scissors 700 is assembled, the pivot 710 is inserted through the hole 705B and the hole 705A. The surface 703B contacts the surface 703A. Depending on how far the surgical scissors 700 are opened, a variable portion of the surface 703A and a variable portion of the surface 703B are shielded from receiving UV-C radiation. The variable portions of the surfaces are represented by the cross-hatched region 710 in FIG. 7B and FIG. 7C. In addition, the region within the hole 705A, the region within the hole 705B, and the surfaces of the pivot 710 in contact with the arm 702A and the arm 702B are shielded from receiving UV-C radiation.

In an embodiment of the invention, at least a portion of the arm 702A about the pivot hole 705A and at least a portion of the arm 702B about the pivot hole 705B are fabricated from material, or materials, substantially transparent to UV-C radiation. In addition, the pivot 710 can be fabricated from material, or materials, substantially transparent to UV-C radiation.

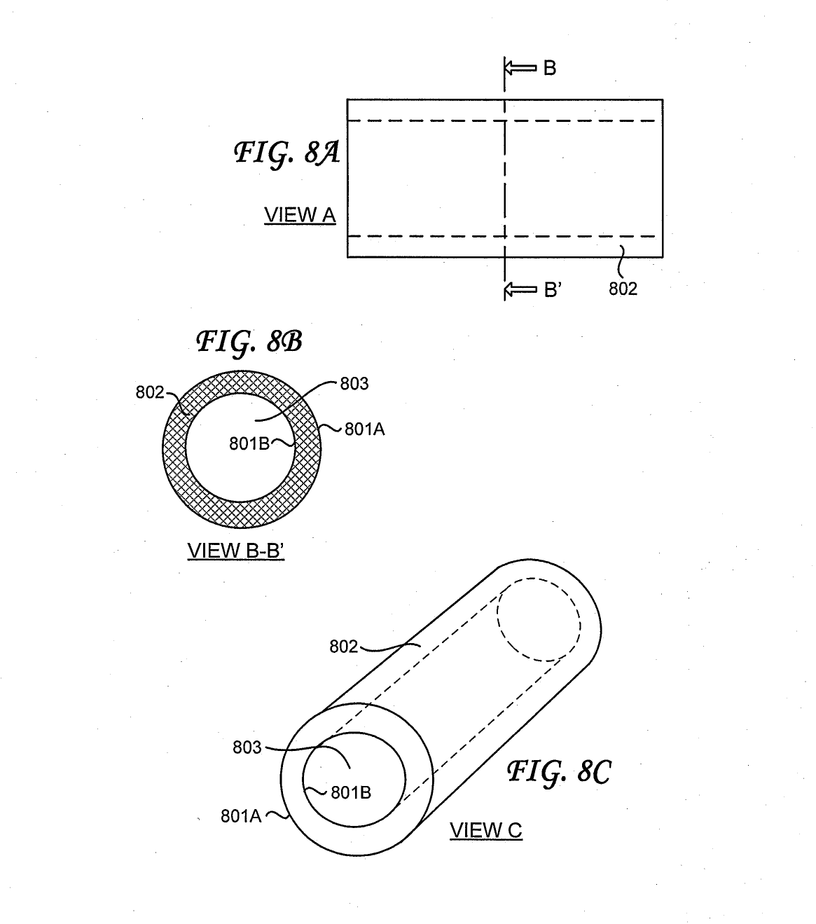

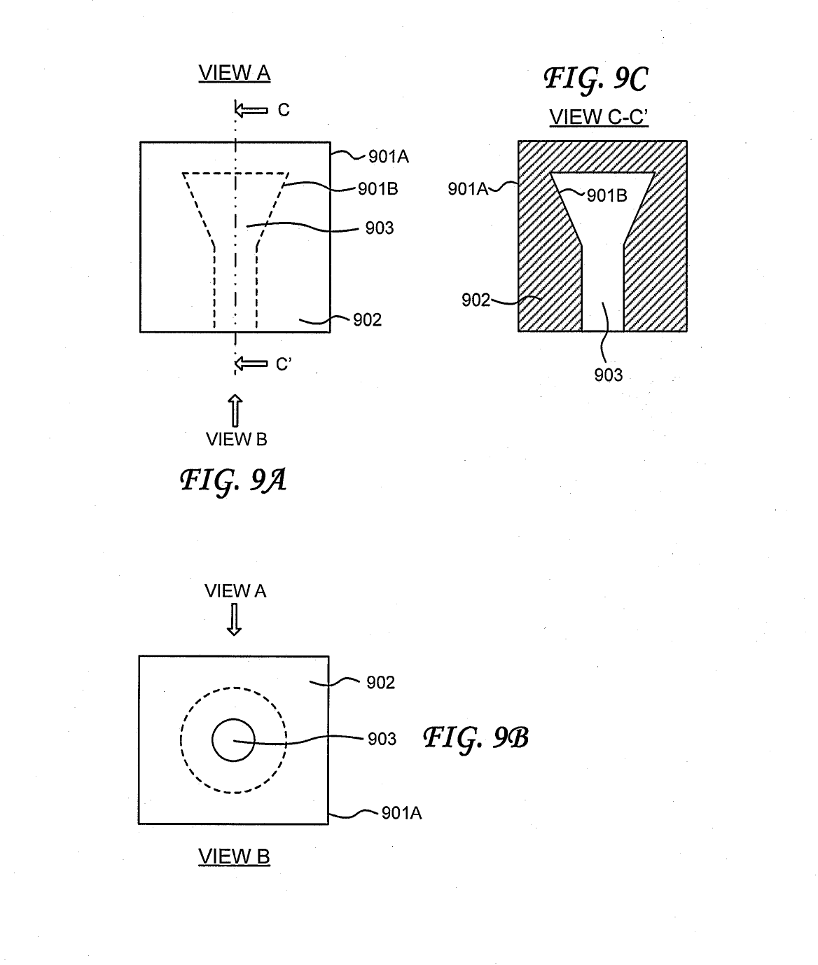

FIG. 8A-FIG. 8C and FIG. 9A-FIG. 9C show examples of objects with internal spaces. In medical terminology, an interior space is referred to as a “lumen”. Examples of medical instruments or devices with lumens include catheters, hollow needles, syringes, and endoscopes.

FIG. 8A-FIG. 8C show schematic views of a tubular structure 802; both ends are open. The tubular structure can represent a portion of a medical instrument or device (or arbitrary object); the tubular structure can also represent an entire device (or arbitrary object), such as a catheter. FIG. 8A shows a side view (View A); FIG. 8B shows a cross-sectional view (View B-B′); and FIG. 8C shows a perspective view (View C). The tubular structure 802, as shown, has a uniform cylindrical geometry with a uniform wall thickness; in general, a tubular structure can have an arbitrary geometry, including irregular geometries that vary along the length of the tubular structure and non-uniform wall thickness. The tubular structure 802 has an exterior surface 801A and an interior surface 801B. The lumen 803 is the space bounded by the interior surface 801B.

In medical instruments or devices, the tubular structure 802 is typically fabricated from a material that shields at least a portion of the interior surface 801B and at least a portion of the lumen 803 from UV-C radiation. In an embodiment of the invention, the tubular structure 802 is fabricated from material, or materials, substantially transparent to UV-C radiation.

FIG. 9A-FIG. 9C show schematics of a block with a cavity closed at one end. The block can represent a portion of, or the entirety of, a medical instrument or device (or arbitrary object). FIG. 9A (View A) and FIG. 9B (View B) show orthogonal views; FIG. 9C (View C-C′) shows a cross-sectional view. The block 902 has an exterior surface 901A. The block 902 has a cavity bounded by the interior surface 901B. The space within the cavity is the lumen 903. In medical instruments or devices, the block 902 is typically fabricated from a material that shields at least a portion of the interior surface 901B and at least a portion of the lumen 903 from UV-C radiation. In an embodiment of the invention, the block 902 is fabricated from material, or materials, substantially transparent to UV-C radiation.

In an embodiment of the invention, other medical supplies, such as sutures (also referred to as suture thread) are fabricated from material, or materials substantially transparent to UV-C radiation such that all portions of the medical supply can be sterilized by UV-C irradiation.

The UV-C irradiation process described above for sterilization of instruments can be advantageously combined with other sterilization processes. Some examples are described below.

The ultraviolet irradiation devices described above can process sterilized, coupon instruments (instruments without lumens) in seconds (total start-to-finish). In an embodiment of the invention, an ultraviolet irradiation device is used in combination with a UV-C hand sterilizer (as described in U.S. Pat. No. 8,142,713). The instrument is not sealed in a pouch or kit; it is placed directly into an ultraviolet irradiation device. An operator wears a glove on his hand, sterilizes the glove with the UV-C hand sterilizer, removes the sterilized instrument from the ultraviolet irradiation device, and hands the sterilized instrument directly to a surgeon, who is also wearing a freshly-sterilized glove. Technical sterilization can be achieved with this process.

Autoclaves can sterilize instruments with lumens since the high temperatures will inactivate the pathogens within the lumen. Autoclaves, furthermore, will sterilize an instrument even when there is a layer of dirt covering live pathogens, since the layer does not block the heat from inactivating all the pathogens. In conventional autoclave processing, warm-up, wrapping, cool-down, and unwrapping take considerable time. Eliminating the wrapping and unwrapping steps can reduce the total autoclave sterilization cycle time to approximately 6 minutes. After autoclave sterilization, however, the surfaces of the instrument can become contaminated through improper handling by the operator or through contact with contaminated room air.

In an embodiment of the invention, the UV-C sterilization process described above is used in combination with autoclave sterilization process. For example, lumen-type instruments without wrapping are first sterilized with a 6-minute cycle, autoclave sterilization process, sealed in a pouch substantially transparent to UV-C radiation, and then surface sterilized with UV-C radiation. Technical sterilization can be achieved with this process.

UV-C sterilization can also be applied advantageously to the production of sterile surgical gloves. Producing surgical gloves that are always sterile when they are removed from the package is a manufacturing challenge and is costly. Currently, sterility of the delivered surgical glove cannot be assured; there is always a small component of the product that is not sterile. The manufacturing process for sterile surgical gloves is not under good control and an uncharacterized fraction of the gloves is not sterile upon removal from their package at the time of use. The manufacturing process can lead to variability in the fraction that is not sterile, depending on the time and conditions under which they are manufactured. Furthermore, the manufacturing process used to semi-reliably achieve sterility adds significantly to the cost of manufacture. Exam gloves are typically not sterile, no attempt is made to achieve sterility, and their cost is substantially less. The only goal of the exam glove is to protect the wearer and that does not require sterility.

In an embodiment of the invention, UV-C sterilization is applied to the manufacture of sterile surgical gloves; the process can produce gloves that are guaranteed sterile in the package. A sealed pouch, substantially-transparent to UV-C radiation, containing one glove or a pair of gloves is subjected during manufacture to UV-C sterilization as described above. In an embodiment, the package is made of TOPAS COC, 1-2 mils thick. The UV-C transmission is virtually 100%, and the TOPAS COC material is sturdy.

Production includes a close-to-final step in which the sealed pouch, containing the glove or gloves, is irradiated as it travels within a high intensity UV-C irradiation tunnel with a total dose sufficient to sterilize with certainty; technical sterilization can be achieved with this process. This process would reduce the cost of producing and packaging a sterile glove and essentially would guarantee sterility as it comes out of the package. In an embodiment of the invention, surgical gloves are fabricated from material, or materials (such as TOPAS COC or Teflon AF), substantially transparent to UV-C radiation.

The above description has focused on UV-C sterilization of surgical instruments. As discussed above, however, an arbitrary “object” can be sterilized with UV-C irradiation. Objects include other medical instruments and devices such as dental instruments, stethoscopes, and cuffs for blood pressure gauges. Objects also include non-medical objects such as eating utensils, food-preparation equipment, and common objects such as toys and cell phones that can be a source of infection. In applications that do not require technical sterilization, the applied UV-C dose can be adjusted to provide a specified percentage of pathogen inactivation.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A method for sterilizing an object, the method comprising the steps of:
inserting the object into a container, wherein at least a portion of the container is substantially transparent to ultraviolet-C radiation having a wavelength from about 235 nm to about 295 nm;
sealing the container; and
irradiating, for an exposure time, the container and the object with ultraviolet-C radiation having a wavelength from about 235 nm to about 295 nm.

2. The method of claim 1, wherein the ultraviolet-C radiation has a wavelength of about 253.7 nm.

3. The method of claim 1, wherein the ultraviolet-C radiation has a predetermined intensity and the exposure time is determined such that the step of irradiating inactivates a predetermined portion of user-specified pathogens disposed on the object prior to the step of irradiating.

4. The method of claim 3, wherein the predetermined portion is 99.9999%.

5. The method of claim 4, wherein:
the ultraviolet-C radiation has a wavelength of about 253.7 nm;
the predetermined intensity is about 500 watts/m²; and
the exposure time is less than about 10 sec.

6. The method of claim 1, wherein the container is a pouch comprising a material selected from the group consisting of:
cyclic olefin copolymer; and
fluoropolymer.

7. The method of claim 1, wherein the container is a kit and at least a portion of the kit comprises a material selected from the group consisting of:
quartz;
borosilicate glass; and
fluoropolymer.

8. The method of claim 1, wherein the object comprises a surgical instrument.

9. A pouch comprising:
a pouch body comprising a material substantially impermeable to transmission of pathogens and substantially transparent to ultraviolet-C radiation having a wavelength from about 235 nm to about 295 nm; and
a pouch opening configured to be sealed such that the pouch, after the pouch opening has been sealed, is substantially impermeable to transmission of pathogens.

10. The pouch of claim 9, wherein the pouch opening is configured to be sealed with adhesive or by thermal fusion.

11. The pouch of claim 9, wherein the pouch opening is configured to be sealed with a mechanical seal.

12. The pouch of claim 9, wherein the ultraviolet-C radiation has a wavelength of about 253.7 nm.

13. The pouch of claim 9, wherein the material is selected from the group consisting of:
cyclic olefin copolymer; and
fluoropolymer.

14. A container comprising:
a receptacle comprising:
a receptacle plate comprising a first material substantially impermeable to transmission of pathogens and substantially transparent to ultraviolet-C radiation having a wavelength from about 235 nm to about 295 nm;
a receptacle side wall comprising a second material substantially impermeable to transmission of pathogens, wherein the receptacle side wall is sealed to the receptacle plate; and
a receptacle opening opposed to the receptacle plate; and
a cover comprising a cover plate comprising a third material substantially impermeable to transmission of pathogens and substantially transparent to ultraviolet-C radiation having a wavelength from about 235 nm to about 295 nm, wherein the cover is configured to mechanically seal the receptacle opening, such that, after the cover has mechanically sealed the receptacle opening:
the cover plate is opposed to the receptacle plate; and
the container is substantially impermeable to transmission of pathogens.

15. The container of claim 14, wherein the cover further comprises a cover side wall comprising a fourth material substantially impermeable to transmission of pathogens, wherein the cover side wall is sealed to the cover plate.

16. The container of claim 14, wherein the ultraviolet-C radiation has a wavelength of about 253.7 nm.

17. The container of claim 14, wherein:
the first material is selected from the group consisting of:
quartz;
borosilicate glass; and
fluoropolymer; and
the third material is selected from the group consisting of:
quartz;
borosilicate glass; and
fluoropolymer.

18. The container of claim 14, wherein the receptacle side wall comprises an interior surface, at least a portion of which comprises a fifth material substantially reflective of ultraviolet-C radiation having a wavelength from about 235 nm to about 295 nm.

19. A container comprising:
a receptacle comprising:
a first receptacle plate comprising a first material substantially impermeable to transmission of pathogens and substantially transparent to ultraviolet-C radiation having a wavelength from about 235 nm to about 295 nm;
a second receptacle plate comprising a second material substantially impermeable to transmission of pathogens and substantially transparent to ultraviolet-C radiation having a wavelength from about 235 nm to about 295 nm, wherein the second receptacle plate is opposed to the first receptacle plate;
a side wall comprising a third material substantially impermeable to transmission of pathogens, wherein the side wall is sealed to the first receptacle plate and sealed to the second receptacle plate; and
a receptacle opening opposed to a portion of the side wall; and
a cover comprising a cover plate comprising a fourth material substantially impermeable to transmission of pathogens, wherein the cover is configured to mechanically seal the receptacle opening, such that, after the cover has mechanically sealed the receptacle opening, the container is impermeable to transmission of pathogens.

20. The container of claim 19, wherein the cover further comprises a cover side wall comprising a fifth material substantially impermeable to transmission of pathogens, wherein the cover side wall is sealed to the cover plate.

21. The container of claim 19, wherein the ultraviolet-C radiation has a wavelength of about 253.7 nm.

22. The container of claim 19, wherein:
the first material is selected from the group consisting of:
quartz;
borosilicate glass; and
fluoropolymer; and
the second material is selected from the group consisting of:
quartz;
borosilicate glass; and
fluoropolymer.

23. The container of claim 19, wherein the receptacle side wall further comprises an interior surface, at least a portion of which comprises a fifth material substantially reflective of ultraviolet-C radiation having a wavelength from about 235 nm to about 295 nm.
